(19) United States
(12) Patent Application Publication
(10) Pub. No.: US 2024/0027450 A1
(43) Pub. Date: Jan. 25, 2024

(54) COMPOSITIONS AND METHODS FOR DIAGNOSING SARS-COV-2 (COVID-19) AND FOR MONITORING SARS-COV-2-SPECIFIC IMMUNOLOGICAL MEMORY

(71) Applicant: QIAGEN SCIENCES LLC, Germantown, MD (US)

(72) Inventors: Jeff BOYLE, Boyds, MD (US); Jenny HOWARD, Boyds, MD (US); Soumya JAGANATHAN, New Market, MD (US); Dave LEWINSOHN, Portland, OR (US); Deborah LEWINSOHN, Portland, OR (US); Gwendolyn SWARBRICK, Gresham, OR (US)

(21) Appl. No.: 18/255,308

(22) PCT Filed: Dec. 3, 2021
(86) PCT No.: PCT/US2021/061875

§ 371 Date: May 31, 2023

Related U.S. Application Data
(60) Provisional application No. 63/121,490, filed on Dec. 4, 2020.
(60) Provisional application No. 63/150,890, filed on Feb. 18, 2021.

(51) Int. Cl. G01N 33/569; G01N 33/68

(52) U.S. Cl. CPC G01N 33/56983; G01N 33/6866; G01N 2333/165; G01N 2469/20; G01N 2333/57

(57) ABSTRACT

Compositions and methods are provided for detection, diagnosis and prognosis of severe acute respiratory syndrome coronavirus 2 (SARS-CoV-2) disease (COVID-19) and for characterization of SARS-CoV-2 antigen-specific T-cell immune responsiveness in COVID-19 patient samples, including in secondary in vitro immune response assays for long-lived anamnestic (memory) T-cell responses. Disclosed compositions and methods include a method that comprises contacting, in vitro, whole blood samples from subjects suspected of having COVID-19 or who have previously been exposed to SARS-CoV-2, with synthetic peptides comprising T-cell epitope-containing regions derived from SARS-CoV-2 Spike proteins; and indirectly detecting SARS-CoV-2-specific activated T-cells by determining production of a T-cell immune response indicator (e.g., interferon-γ) in response to stimulation by the Spike protein-derived peptides.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 830109_428_Sequence_Listing.txt. The text file is 25 KB, was created on Dec. 3, 2021, and is being submitted electronically via EFS-Web.

### BACKGROUND

### Technical Field

The present disclosure relates generally to compositions and methods for diagnosing severe acute respiratory syndrome coronavirus 2 (SARS-CoV-2) disease (coronavirus disease 2019, COVID-19) and for detecting antigen-specific T cell-mediated immune responses to SARS-CoV-2. More specifically, there are provided combinations of multiple T-cell epitope-containing peptides derived from SARS-CoV-2 Spike polypeptide antigens, for use in a sensitive secondary in vitro immune response assay for SARS-CoV-2-specific T-cell responsiveness.

### Description of the Related Art

The novel severe acute respiratory syndrome coronavirus 2 (SARS-CoV-2) is responsible for the coronavirus disease 2019 (COVID-19) pandemic that swept the world in 2020, with a still-growing toll (as of February 2021) of over 107 million cases and over 2.3 million deaths reported worldwide. SARS-CoV-2 is an enveloped virus that gains entry to host cells by multiple mechanisms, including by interactions between viral surface-disposed peplomers (protein spikes), which comprise assembled homotrimers of the posttranslationally processed SARS-CoV-2 Spike glycoprotein (SARS2 Spike, UNIPROT P0DTC2), and widely expressed cell surface angiotensin converting enzyme 2 (ACE2) receptors (Hikmet et al., 2020 Mol. Syst. Biol. 16(7): e9610). Spike-ACE2 binding is mediated by a receptor binding domain (RBD) that resides in the structure formed by amino acids 319-541 of the full-length Spike protein (SARS2 Spike, UNIPROT P0DTC2) (Shang et al., 2020 Nature 581:221-224).

The symptoms and severity of, and susceptibility to, COVID-19 vary tremendously among infected humans, as also do the qualitative and quantitative aspects of the human immune response to SARS-CoV-2. Humoral (antibody) and T cell-mediated responses of highly variable degrees of magnitude and duration have been reported (e.g., Stephens et al., 2020 JAMA 324:1279-1281; Schulien et al., 2020 Nature Med. PMID: 33184509; Chen et al., 2020 Nat Rev Immunol July 29: 1-8 41577_2020_Article_402 PMID 32728222; Gimenez et al., 2020 J Med Virol July 2 10.1002/jmv.26213 PMID 32579268; Hellerstein, 2020 Vaccine X6:100076). Numerous unanswered questions remain with respect to anti-SARS-CoV-2 immune responses, including the specificity and efficacy of adaptive immunity and the persistence of immunological protection against reinfection.

For example, detection of active SARS-CoV-2 infections have typically involved polymerase chain reaction (PCR) amplification of viral RNA in a patient sample, such as a nasal swab, saliva, bronchial fluid, or respiratory sputum sample. False-negative PCR tests may result from low viral loads, effective clearance of the infection by the time the sample is collected, and/or artifacts of the sample collection and assay procedures. Exposure of an individual to the coronavirus may be also detected by testing a patient blood sample for the presence of antibodies that specifically bind to one or more SARS-CoV-2 antigens, such as the Spike glycoprotein, the nucleocapsid phosphoprotein, or other viral antigens. Antibodies may not be detectable until one to three weeks post-infection, and the antibody isotype (e.g., IgM or IgG) may suggest the stage of maturation of the humoral immune response. Fluctuations in the antigen-specificities, isotypes, titers, and persistence in the circulation of SARS-CoV-2-specific antibodies have hindered the reliability and information content of such antibody tests for characterization of the immunological history of a COVID-19 patient.

Clearly there remains a need for improved diagnostic and prognostic assessment of COVID-19 and for evaluation of the durability of SARS-CoV-2-specific adaptive immune responsiveness, including assessment of patient samples for potential amnestic (memory) immunological responses. The presently disclosed invention embodiments address these needs and offer other related advantages.

### BRIEF SUMMARY

According to certain embodiments of the invention that is disclosed herein, there is provided a composition for diagnosis or prognosis of coronavirus disease 2019 (Covid-19), or for detecting an antigen-specific T cell-mediated immune response to severe acute respiratory syndrome coronavirus 2 (SARS-CoV-2), comprising 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 and 39, preferably in 1, 2, 3, 4, 5, 6, 7, 8, 9, 10, 11, 12, 13, 14, 15, 16, 17, 18, 19, 20, or 21 of SEQ ID NOS: 1-20 and 39:

SEQ ID NO: 1
YPDKVFRSSVLHST,
SEQ ID NO: 2
VLHSTQDLFLPFF,
SEQ ID NO: 3
KSWMESEFRVY,
SEQ ID NO: 4
RVYSSANNCTFEY,
SEQ ID NO: 5
EFVFKNIDGYFK,
SEQ ID NO: 6
YYVGYLQPRTFLLKY,
SEQ ID NO: 7
EVFNATRFASVYAW,
SEQ ID NO: 8
RISNCVADYSVLYN,
SEQ ID NO: 9
YSVLYNSASFTFKCY,
SEQ ID NO: 10
CFTNVYADSFV,
SEQ ID NO: 11
LYRLFRKSNLKPF,
SEQ ID NO: 12
YQPYRVVVLSFEL,
SEQ ID NO: 13
WRVYSTGSNVFQ,
SEQ ID NO: 14
TNSPRRARSVASQSI,
SEQ ID NO: 15
RSVASQSIIAYTMSL,
SEQ ID NO: 16
MTKTSVDCTMY,
SEQ ID NO: 17
PLLTDEMIAQYTSALL,
SEQ ID NO: 18
AALQIPFAMQMAYRF,
SEQ ID NO: 19
RAAEIRASANLAATKM,
SEQ ID NO: 20
KYEQYIKWPWYIWLGFI,
SEQ ID NO: 21
YIWLGFIAGLIAIVM,
and
SEQ ID NO: 39
YHLMSFPQSAPH,

or one or more variants thereof having at least 80% amino acid sequence identity to the amino acid sequences set forth in SEQ ID NOS:1-21 and 39.

In certain embodiments the composition further comprises 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36:

SEQ ID NO: 22
RVQPTESIVRFPNITNLCPFGEVEN,
SEQ ID NO: 23
NLCPFGEVFNATRFASVYAWNRKRI,
SEQ ID NO: 24
SVYAWNRKRISNCVADYSVLYNSAS,
SEQ ID NO: 25
DYSVLYNSASFSTFKCYGVSPTKLN,
SEQ ID NO: 26
CYGVSPTKLNDLCFTNVYADSFVIR,
SEQ ID NO: 27
NVYADSFVIRGDEVRQIAPGQTGKI,
SEQ ID NO: 28
RQIAPGQTGKIADYNYKLPDDFTGC,
SEQ ID NO: 29
YKLPDDFTGCVIAWNSNNLDSKVGG,
SEQ ID NO: 30
SNNLDSKVGGNYNYLYRLFRKSNLK,
SEQ ID NO: 31
YRLFRKSNLKPFERDISTEIYQAGS,
SEQ ID NO: 32
ISTEIYQAGSTPCNGVEGFNCYFPL,
SEQ ID NO: 33
VEGFNCYFPLQSYGFQPTNGVGYQP,
SEQ ID NO: 34
FQPTNGVGYQPYRVVVLSFELLHAP,
SEQ ID NO: 35
VLSFELLHAPATVCGPKKSTNLVKN,
and
SEQ ID NO: 36
PKKSTNLVKNKCVNF,

or one or more variants thereof having at least 80% amino acid sequence identity to the amino acid sequences set forth in SEQ ID NOS:22-36.

In certain embodiments there is provided a composition for diagnosis or prognosis of coronavirus disease 2019 (Covid-19), or for detecting an antigen-specific T cell-mediated immune response to severe acute respiratory syndrome coronavirus 2 (SARS-CoV-2), comprising a first set of 21 isolated oligopeptides that comprise the amino acid sequences set forth in SEQ ID NOS: 1-21 and 39, preferably SEQ ID NOS: 1-20 and 39, or one or more variants thereof having at least 80% amino acid sequence identity to the amino acid sequences set forth in one or more of SEQ ID NOS:1-21 and 39, preferably SEQ ID NOS: 1-20 and 39.

In certain embodiments the composition further comprises a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

In certain embodiments there is provided a composition for diagnosis or prognosis of coronavirus disease 2019 (Covid-19), or for detecting an antigen-specific T cell-mediated immune response to severe acute respiratory syndrome coronavirus 2 (SARS-CoV-2), comprising 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36, or one or more variants thereof having at least 80% amino acid sequence identity to the amino acid sequences set forth in SEQ ID NOS:22-36.

In certain embodiments there is provided a composition for diagnosis or prognosis of coronavirus disease 2019 (Covid-19), or for detecting an antigen-specific T cell-mediated immune response to severe acute respiratory syndrome coronavirus 2 (SARS-CoV-2), comprising a set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS:22-36, and that each comprise a SARS-CoV-2 Spike protein receptor binding domain (RBD) CD4+ T-cell epitope.

In another embodiment there is provided a method for detecting SARS-CoV-2 spike protein antigen-specific cell-mediated immune response activity in a biological sample from a subject, comprising (a) incubating in vitro an incubation test mixture that comprises (i) a biological sample comprising T-cells and antigen-presenting cells from the subject admixed and (ii) a first peptide composition comprising a first set of 21 isolated oligopeptides that comprise the amino acid sequences set forth in SEQ ID NOS: 1-21 or that comprise the amino acid sequences set forth in SEQ ID NOS: 1-20 and 39, or one or more variants thereof having at least 80% amino acid sequence identity to the amino acid sequences set forth in one or more of SEQ ID NOS:1-21 and preferably in one or more of SEQ ID NOS: 1-20 and 39, under conditions and for a time sufficient for specific recognition by said T-cells of a SARS-CoV-2 spike protein T-cell epitope that is present in said first composition to stimulate generation of a T-cell immune response indicator; and (b) detecting a first level of the T-cell immune response indicator in the incubation test mixture, wherein presence of SARS-CoV-2 spike protein antigen-specific cell-mediated immune response activity in the biological sample is indicated by detection in (b) of said first level of the T-cell immune response indicator that is increased relative to a control level of the T-cell immune response indicator obtained by incubating the biological sample in a control incubation without the peptide composition, and thereby detecting SARS-CoV-2 spike protein antigen-specific cell-mediated immune response activity.

In certain further embodiments the incubation test mixture further comprises (iii) a second peptide composition comprising a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

In certain embodiments, the biological sample is obtained from the subject before, after, or both before and after a SARS-CoV-2 vaccine has been administered to the subject.

In certain embodiments of the above described methods, the biological sample comprises at least one of whole blood, sputum, pulmonary lavage fluid, or lymph. In certain other embodiments the biological sample comprises at least one of (a) whole blood, (b) a cellular fraction of whole blood, (c) isolated peripheral blood white cells, or (d) isolated peripheral blood mononuclear cells. In certain embodiments the T-cell immune response indicator is interferon-gamma (IFN-γ), and in certain further embodiments the IFN-γ is soluble IFN-γ released by the T-cells.

In certain embodiments of the above described methods, the T-cell immune response indicator comprises at least one of T-cell proliferation and expression of a T-cell cytokine, which in certain further embodiments is selected from IL-1α, IL-1β, IL-2, IL-10, IL-12, IL-17, TNF-α, TNF-β, and IFN-γ. In certain embodiments expression of the T-cell cytokine is detected as soluble T-cell cytokine released by the T-cells, and in certain further embodiments the T-cell cytokine is selected from IL-1α, IL-1β, IL-2, IL-10, IL-12, IL-17, TNF-α, TNF-β, and IFN-γ. In certain embodiments of the above described methods, the T-cell cytokine is detected by determining detectable specific binding of a binding agent to the T-cell cytokine. In certain embodiments the binding agent comprises at least one antibody that binds specifically to the T-cell cytokine. In certain further embodiments the at least one antibody is selected from a monoclonal antibody and a polyclonal antibody. In certain embodiments the at least one antibody is immobilized on a solid phase.

Turning to certain other presently disclosed embodiments, there is provided a composition that is selected from a first nucleic acid composition and a second nucleic acid composition: (I) the first nucleic acid composition comprising one or a plurality of isolated nucleic acid molecules that encode 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 and 39, preferably in SEQ ID NOS: 1-20 and 39, or one or more variants thereof having at least 80% amino acid sequence identity to the amino acid sequences set forth in SEQ ID NOS:1-21 and 39, preferably SEQ ID NOS: 1-20 and 39, wherein the isolated oligopeptides, after being contacted with a whole blood sample obtained from a subject who has previously been infected with SARS-CoV-2, are capable of eliciting a secondary in vitro immune response by T-cells in the whole blood sample; and (II) the second nucleic acid composition comprising one or a plurality of isolated nucleic acid molecules that encode 21 isolated oligopeptides that comprise the amino acid sequences set forth in SEQ ID NOS: 1-21 or in SEQ ID NOS: 1-20 and 39, or one or more variants thereof having at least 80% amino acid sequence identity to the amino acid sequences set forth in one or more of SEQ ID NOS:1-21 and 39, wherein the isolated oligopeptides, after being contacted with a whole blood sample obtained from a subject who has previously been infected with SARS-CoV-2, are capable of eliciting a secondary in vitro immune response by T-cells in the whole blood sample.

In another embodiment there is provided a composition that is selected from a first nucleic acid composition and a second nucleic acid composition: (I) the first nucleic acid composition comprising one or a plurality of isolated nucleic acid molecules that encode 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36 or one or more variants thereof having at least 80% amino acid sequence identity to the amino acid sequences set forth in SEQ ID NOS:22-36, wherein the isolated oligopeptides, after being contacted with a whole blood sample obtained from a subject who has previously been infected with SARS-CoV-2, are capable of eliciting a secondary in vitro immune response by T-cells in the whole blood sample; and (II) the second nucleic acid composition comprising one or a plurality of isolated nucleic acid molecules that encode 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS:22-36, wherein the isolated oligopeptides, after being contacted with a whole blood sample obtained from a subject who has previously been infected with SARS-CoV-2, are capable of eliciting a secondary in vitro immune response by T-cells in the whole blood sample.

Certain embodiments provide a vector composition comprising one or more nucleic acid vectors that comprise one or more of the above described nucleic acid compositions. Certain other embodiments provide a host cell comprising such a vector composition.

In certain other embodiments of the herein described composition for diagnosis or prognosis of coronavirus disease 2019 (Covid-19), or for detecting an antigen-specific T cell-mediated immune response to severe acute respiratory syndrome coronavirus 2 (SARS-CoV-2), the 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprise at least: (a) the amino acid sequences set forth in SEQ ID NOS: 1, 3, and 5, or one or more variants having at least 80% amino acid sequence identity thereto; (b) the amino acid sequences set forth in SEQ ID NOS: 2, 10, and 12, or one or more variants having at least 80% amino acid sequence identity thereto; (c) the amino acid sequences set forth in SEQ ID NOS: 3, 6, and 11, or one or more variants having at least 80% amino acid sequence identity thereto; or (d) the amino acid sequences set forth in SEQ ID NOS: 4, 19, and 20, or one or more variants having at least 80% amino acid sequence identity thereto.

In certain other embodiments of the herein described composition for diagnosis or prognosis of coronavirus disease 2019 (Covid-19), or for detecting an antigen-specific T cell-mediated immune response to severe acute respiratory syndrome coronavirus 2 (SARS-CoV-2), the 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprise at least: (a) the amino acid sequences set forth in SEQ ID NOS: 1, 3, and 5, or one or more variants having at least 80% amino acid sequence identity thereto; (b) the amino acid sequences set forth in SEQ ID NOS: 2, 4, and 7, or one or more variants having at least 80% amino acid sequence identity thereto; (c) the amino acid sequences set forth in SEQ ID NOS: 8, 10, and 12, or one or more variants having at least 80% amino acid sequence identity thereto; (d) the amino acid sequences set forth in SEQ ID NOS: 9, 14, and 15, or one or more variants having at least 80% amino acid sequence identity thereto; (e) he amino acid sequences set forth in SEQ ID NOS: 6, 11, and 18, or one or more variants having at least 80% amino acid sequence identity thereto; (f) the amino acid sequences set forth in SEQ ID NOS: 13, 16, and 39, or one or more variants having at least 80% amino acid sequence identity thereto; or (g) the amino acid sequences set forth in SEQ ID NOS: 17, 19, and 20, or one or more variants having at least 80% amino acid sequence identity thereto.

Turning to another embodiment there is provided a method for detecting SARS-CoV-2 spike protein antigen-specific cell-mediated immune response activity in a biological sample from a subject, comprising (a) incubating in vitro an incubation test mixture that comprises (i) a biological sample comprising T-cells and antigen-presenting cells from the subject admixed and (ii) a first peptide composition comprising 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 or in one of SEQ ID NOS: 1-20 and 39, or one or more variants thereof having at least 80% amino acid sequence identity to the amino acid sequences set forth in SEQ ID NOS:1-21 or in SEQ ID NOS: 1-20 and 39, under conditions and for a time sufficient for specific recognition by said T-cells of a SARS-CoV-2 spike protein T-cell epitope that is present in said first composition to stimulate generation of a T-cell immune response indicator; and (b) detecting a first level of the T-cell immune response indicator in the incubation test mixture, wherein presence of SARS-CoV-2 spike protein antigen-specific cell-mediated immune response activity in the biological sample is indicated by detection in (b) of said first level of the T-cell immune response indicator that is increased relative to a control level of the T-cell immune response indicator obtained by incubating the biological sample in a control incubation without the peptide composition, and thereby detecting SARS-CoV-2 spike protein antigen-specific cell-mediated immune response activity.

In certain further embodiments the incubation test mixture further comprises (iii) a second peptide composition comprising a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

In certain further embodiments of the herein described methods, the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprises at least: (a) the amino acid sequences set forth in SEQ ID NOS: 1, 3, and 5 or one or more variants having at least 80% amino acid sequence identity thereto; (b) the amino acid sequences set forth in SEQ ID NOS: 2, 10, and 12 or one or more variants having at least 80% amino acid sequence identity thereto; (c) the amino acid sequences set forth in SEQ ID NOS: 3, 6, and 11 or one or more variants having at least 80% amino acid sequence identity thereto; or (d) the amino acid sequences set forth in SEQ ID NOS: 4, 19, and 20 or one or more variants having at least 80% amino acid sequence identity thereto.

In certain further embodiments of the herein described methods, the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprises at least: (a) the amino acid sequences set forth in SEQ ID NOS: 1, 3, and 5 or one or more variants having at least 80% amino acid sequence identity thereto; (b) the amino acid sequences set forth in SEQ ID NOS: 2, 4, and 7 or one or more variants having at least 80% amino acid sequence identity thereto; (c) the amino acid sequences set forth in SEQ ID NOS: 8, 10, and 12 or one or more variants having at least 80% amino acid sequence identity thereto; (d) the amino acid sequences set forth in SEQ ID NOS: 9, 14, and 15 or one or more variants having at least 80% amino acid sequence identity thereto; (e) the amino acid sequences set forth in SEQ ID NOS: 6, 11, and 18 or one or more variants having at least 80% amino acid sequence identity thereto; (f) the amino acid sequences set forth in SEQ ID NOS: 13, 16, and 39 or one or more variants having at least 80% amino acid sequence identity thereto; or (g) the amino acid sequences set forth in SEQ ID NOS: 17, 19, and 20 or one or more variants having at least 80% amino acid sequence identity thereto.

In certain further embodiments of the herein described methods, the biological sample is obtained from the subject before, after, or before and after a SARS-CoV-2 vaccine has been administered to the subject. In certain embodiments the biological sample comprises at least one of whole blood, sputum, pulmonary lavage fluid, or lymph. In certain embodiments the biological sample comprises at least one of (a) whole blood, (b) a cellular fraction of whole blood, (c) isolated peripheral blood white cells, or (d) isolated peripheral blood mononuclear cells. In certain embodiments the T-cell immune response indicator is interferon-gamma (IFN-γ), which in certain further embodiments is soluble IFN-γ released by the T-cells. In certain embodiments the T-cell immune response indicator comprises at least one of T-cell proliferation and expression of a T-cell cytokine. In certain further embodiments the T-cell cytokine is selected from IL-1α, IL-1β, IL-2, IL-10, IL-12, IL-17, TNF-α, TNF-β, and IFN-γ. In certain embodiments expression of the T-cell cytokine is detected as soluble T-cell cytokine released by the T-cells. In certain further embodiments the T-cell cytokine is selected from IL-1α, IL-1β, IL-2, IL-10, IL-12, IL-17, TNF-α, TNF-β, and IFN-γ.

In certain embodiments of the herein described methods the T-cell cytokine is detected by determining detectable specific binding of a binding agent to the T-cell cytokine. In certain further embodiments the binding agent comprises at least one antibody that binds specifically to the T-cell cytokine. In certain still further embodiments the at least one antibody is selected from a monoclonal antibody and a polyclonal antibody. In certain embodiments the at least one antibody is immobilized on a solid phase.

These and other aspects and embodiments of the invention will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet, are incorporated herein by reference in their entirety, as if each was incorporated individually. Aspects and embodiments of the invention can be modified, if necessary, to employ concepts of the various patents, applications and publications to provide yet further embodiments of the invention.

### BRIEF DESCRIPTION OF THE DRAWINGS

FIG. 1 shows the amino acid sequence of SARS-CoV-2 Spike glycoprotein [SEQ ID NO: 37] (Ref. https://www.uniprot.org/uniprot/P0DTC2).

FIG. 2 shows the amino acid sequence of the Receptor Binding Domain (RBD) [SEQ ID NO: 38] of SARS-CoV-2 Spike glycoprotein.

FIG. 3 shows CD8+ T-cell epitope-containing SARS-CoV-2-Spike protein-derived peptides (SEQ ID NOS: 1-21 and 39) identified using the binding prediction tool NetMHC 4.0; “X” indicates predicted binding to the indicated major histocompatibility complex (MHC) allele (HLA system).

FIG. 4 shows a plot of interferon-gamma (IFNγ) international units per milliliter induced by SARS-CoV-2 spike peptides, less background levels induced when spike peptides were omitted (IU/mL-Nil). IU/mL-Nil values are shown for incubations of whole blood samples from COVID-19 positive and negative donors with only CD4 T cell-inducing RBD peptides, only CD8 T cell-inducing spike peptides, and the combination of CD4 peptides-plus-CD8 peptides. IFN-γ was detected after secondary in vitro T-cell activation with a peptide pool (SEQ ID NOS:1-20 and 39) in whole blood samples collected from previously confirmed SARS-CoV-2 positive and negative donors. Test incubation mixtures were formed by contacting aliquots of the whole blood samples either with the herein described CD8+ T-cell epitope-containing SARS-CoV-2 Spike protein-derived peptides (SEQ ID NOS: 1-20 and 39) alone, or with overlapping CD4+ T-cell epitope-containing SARS-CoV-2 Spike receptor binding domain (RBD) peptides (SEQ ID NOS: 22-36) alone, or with the combination of CD4 and CD8 peptides (SEQ ID NOS: 1-20, 22-36, 39). Following stimulation of T-cells in the incubation admixtures by incubation at 37±1° C., the resulting plasma was harvested and T-cell mediated responses to the SARS-CoV-2 spike protein-derived peptide sets were assessed through the measurement of the resulting cytokine (IFN-γ) that was released. The IFN-γ that was produced was measured using the QuantiFERON® (QIAGEN, Germantown, MD) enzyme-linked immunosorbent assay (ELISA) according to the manufacturer's recommendations. The results for the IU/mL of the IFN-γ after Nil (background) subtraction are shown.

FIG. 5 shows mean values (IU/mL) after Nil (background) subtraction of IFN-γ that was detected following in vitro stimulation, by herein described SARS-CoV-2 peptide antigen compositions, of immune cells in whole blood samples obtained from seven human subjects following administration of SARS-CoV-2 vaccine.

FIG. 6 shows the IFN-γ response (IU/mL-Nil) to CD8+ epitopes alone (SEQ ID NOS: 1-20 and 39; Group 3), CD4 epitopes alone (Ag1, SEQ ID NOS: 22-36) and CD4+-plus-CD8+ epitopes (Ag2, SEQ ID NOS: 1-20, 22-36, and 39) in QuantiFERON® tubes from 14 donors.

FIG. 7 shows mean IFN-γ IU/mL responses for T cell-containing samples from donors 6 and 13 to herein described SARS-CoV-2 Spike protein CD8+ T cell epitope containing peptide Sub-Pool 1 (SEQ ID NOS: 1, 3, 5) alone or in combination with additional SARS-CoV-2 Spike protein-derived peptides.

FIG. 8 shows mean IFN-γ IU/mL responses for T cell-containing samples from donors 1, 3, and 12 to herein described SARS-CoV-2 Spike protein CD8+ T cell epitope containing peptide Sub-Pool 2 (SEQ ID NOS: 2, 10, 12) alone or in combination with additional SARS-CoV-2 Spike protein-derived peptides.

FIG. 9 shows mean IFN-γ IU/mL responses for T cell-containing samples from donors 1 and 6 to herein described SARS-CoV-2 Spike protein CD8+ T cell epitope containing peptide Sub-Pool 3 (SEQ ID NOS: 3, 6, 11) alone or in combination with additional SARS-CoV-2 Spike protein-derived peptides.

FIG. 10 shows mean IFN-γ IU/mL responses for T cell-containing samples from donors2, 3, 6, and 13 to herein described SARS-CoV-2 Spike protein CD8+ T cell epitope containing peptide Sub-Pool 4 (SEQ ID NOS: 4, 19, alone or in combination with additional SARS-CoV-2 Spike protein-derived peptides.

### BRIEF DESCRIPTION OF THE SEQUENCES

SEQ ID NOS: 1-21 and 39 are amino acid sequences (single letter code) of oligopeptides containing SARS-CoV-2 Spike S1 and S2 region CD8+ T-cell epitopes. (The amino acid sequence position numbers from reference sequence SEQ ID NO: 37 are in parentheses).

SEQ ID NO: 1
YPDKVFRSSVLHST (38-51)
SEQ ID NO: 2
VLHSTQDLFLPFF (47-59)
SEQ ID NO: 3
KSWMESEFRVY (150-160)
SEQ ID NO: 4
RVYSSANNCTFEY (158-170)
SEQ ID NO: 5
EFVFKNIDGYFK (191-202)
SEQ ID NO: 6
YYVGYLQPRTFLLKY (265-279)
SEQ ID NO: 7
EVFNATRFASVYAW (340-353)
SEQ ID NO: 8
RISNCVADYSVLYN (357-370)
SEQ ID NO: 9
YSVLYNSASFTFKCY (365-380)
SEQ ID NO: 10
CFTNVYADSFV (391-401)
SEQ ID NO: 11
LYRLFRKSNLKPF (452-464)
SEQ ID NO: 12
YQPYRVVVLSFEL (505-517)
SEQ ID NO: 13
WRVYSTGSNVFQ (633-644)
SEQ ID NO: 14
TNSPRRARSVASQSI (678-692)
SEQ ID NO: 15
RSVASQSIIAYTMSL (685-699)
SEQ ID NO: 16
MTKTSVDCTMY (731-741)
SEQ ID NO: 17
PLLTDEMIAQYTSALL (863-878)
SEQ ID NO: 18
AALQIPFAMQMAYRF (892-906)
SEQ ID NO: 19
RAAEIRASANLAATKM, (1014-1029)
SEQ ID NO: 20
KYEQYIKWPWYIWLGFI (1205-1221)
SEQ ID NO: 21
YIWLGFIAGLIAIVM (1215-1229)
SEQ ID NO: 39
YHLMSFPQSAPH (1047-1058)

SEQ ID NOS: 22-36 are amino acid sequences (single letter code) of oligopeptides containing SARS-CoV-2 Spike RBD CD4+ T-cell epitopes. (The amino acid sequence position numbers from reference sequence SEQ ID NO: 37 are in parentheses).

SEQ ID NO: 22
RVQPTESIVRFPNITNLCPFGEVEN 319-343
SEQ ID NO: 23
NLCPFGEVFNATRFASVYAWNRKRI 334-358
SEQ ID NO: 24
SVYAWNRKRISNCVADYSVLYNSAS 349-373
SEQ ID NO: 25
DYSVLYNSASFSTFKCYGVSPTKLN 364-388
SEQ ID NO: 26
CYGVSPTKLNDLCFTNVYADSFVIR 379-403
SEQ ID NO: 27
NVYADSFVIRGDEVRQIAPGQTGKI 394-418
SEQ ID NO: 28
RQIAPGQTGKIADYNYKLPDDFTGC 408-432
SEQ ID NO: 29
YKLPDDFTGCVIAWNSNNLDSKVGG 423-447
SEQ ID NO: 30
SNNLDSKVGGNYNYLYRLFRKSNLK 438-462
SEQ ID NO: 31
YRLFRKSNLKPFERDISTEIYQAGS 453-477
SEQ ID NO: 32
ISTEIYQAGSTPCNGVEGFNCYFPL 468-492
SEQ ID NO: 33
VEGFNCYFPLQSYGFQPTNGVGYQP 483-507
SEQ ID NO: 34
FQPTNGVGYQPYRVVVLSFELLHAP 497-521
SEQ ID NO: 35
VLSFELLHAPATVCGPKKSTNLVKN 512-536
SEQ ID NO: 36
PKKSTNLVKNKCVNF 527-541

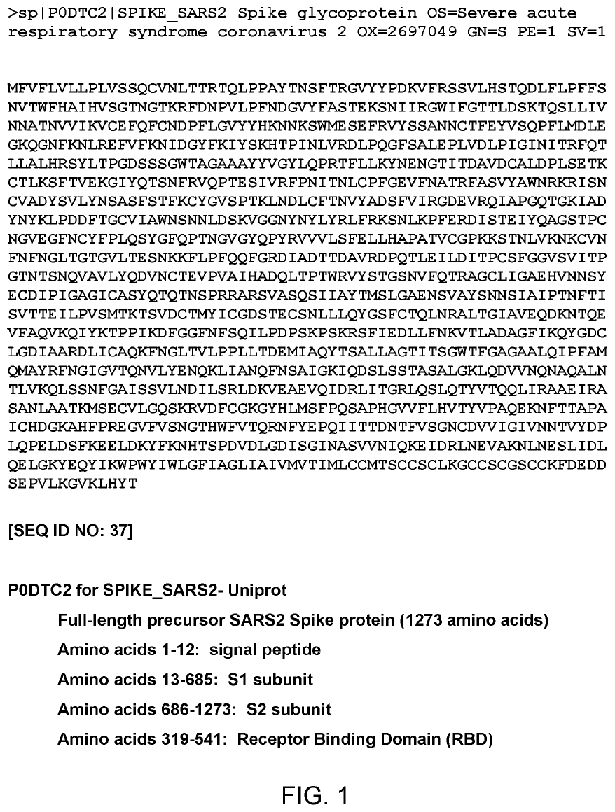

SEQ ID NO: 37 is presented in FIG. 1 and shows the reference amino acid sequence (1273 amino acids) of the full-length precursor of SARS-CoV-2 Spike glycoprotein (www_dot_uniprot.org/uniprot/P0DTC2).

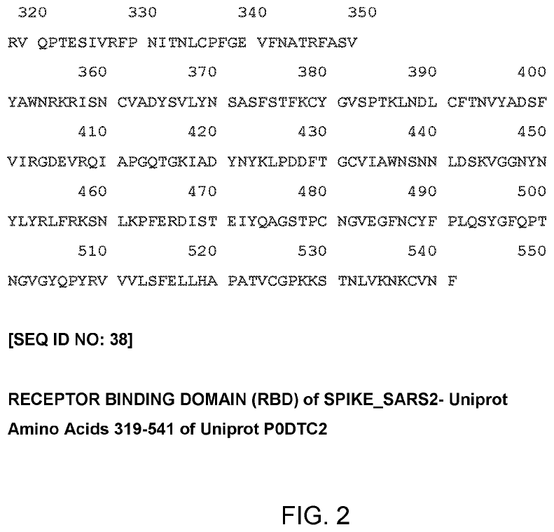

SEQ ID NO: 38 is presented in FIG. 2 and shows the receptor binding domain (RBD), amino acids 319-541, of the SARS-CoV-2 Spike glycoprotein (SPIKE_SARS2-Uniprot).

### DETAILED DESCRIPTION

The presently disclosed invention embodiments relate to artificial compositions and their uses in methods that permit surprisingly sensitive diagnosis and prognosis of coronavirus disease 2019 (COVID-19), and unexpectedly sensitive detection of secondary in vitro CD8+ T cell-mediated immune responses to severe acute respiratory syndrome coronavirus 2 (SARS-CoV-2).

The herein disclosed compositions include multiple oligopeptides comprising CD8+ T-cell epitopes of SARS-CoV-2 spike protein, and sets of such oligopeptides, that comprise a non-naturally occurring combination of defined peptides. As described herein, there is thus provided a composition comprising a peptide or combination of peptides that includes one or more of the 21 SARS-CoV-2 spike protein peptides comprising the amino acid sequences set forth in SEQ ID NOS: 1-21, or preferably the 21 SARS-CoV-2 spike protein peptides comprising the amino acid sequences set forth in SEQ ID NOS: 1-20 and 39, or one or more variants thereof having at least 80% amino acid sequence identity to the amino acid sequences set forth in SEQ ID NOS: 1-21 or preferably SEQ ID NOS: 1-20 and 39. According to certain embodiments, the secondary in vitro CD8+ T cell-mediated immune responses to severe acute respiratory syndrome coronavirus 2 (SARS-CoV-2) that are stimulated by one or more of the oligopeptides of SEQ ID NOS: 1-21 or preferably SEQ ID NOS: 1-20 and 39 may be enhanced by inclusion in the secondary in vitro immune response incubation admixture of one or more of the CD4+ T-cell epitope-containing SARS-CoV-2 spike protein receptor binding domain (RBD) oligopeptides comprising the amino acid sequences set forth in SEQ ID NOS: 22-36, or one or more variants thereof having at least 80% amino acid sequence identity to the amino acid sequences set forth in SEQ ID NO: 22-36.

Related methods will find uses in the detection of SARS-CoV-2 antigen-specific cell-mediated immune response activity in T lymphocyte-containing biological samples from subjects in whom evidence of an active coronavirus infection may be undetectable by conventional nucleic acid-based (e.g., PCR) or serological (e.g., antibody) SARS-CoV-2 diagnostic assays, for example, when a case of COVID-19 has resolved such that the virus has been cleared and protective antibody levels have subsided. Certain embodiments thus contemplate detection of long-lived CD8+ memory T-cells, for example, to assess a subject's immunological ability to mount specific resistance against SARS-CoV-2 reinfection. Certain other embodiments contemplate detection of T-cell mediated immune response capability in T lymphocyte-containing samples obtained from individuals to whom a SARS-CoV-2 vaccine has been administered.

As described herein, SARS-CoV-2 spike protein S1 and S2 region peptides recognized by CD8+ T-cells were identified based on in silico modeling of short peptide binding to major histocompatibility complex (MHC) class I molecules, which are known to provide the immunological context in which non-self antigens are presented to CD8+ T-cells, including CD8+ memory cells and cytotoxic lymphocytes (CTL). Class I binding of putative T-cell epitope-containing oligopeptides of 11-17 amino acids in length was modeled using the binding prediction tool NetMHC 4.0. Such epitopes were identified in spike protein S1 and S2 region peptide sequences from two strains of SARS-CoV-2, the Wuhan and Bei strains, and the selected peptides (Table 1, infra) were found to exhibit 99% amino acid sequence conservation between these two strains while possessing less than 50% sequence homology with polypeptide sequences found in the common human seasonal coronaviruses (HCoV) 229E, NL63, OC43, and HKU1

The presently described combinations of peptides do not occur naturally, given that the MHC class I binding profiles of SEQ ID NOS: 1-21 and 39 reflect differential binding preferences for different class I alleles (Table 1) and so would not naturally be concomitantly presented to an infected host's immune system. The presently disclosed peptide compositions may also contain artificial peptides that differ from the naturally processed SARS-CoV-2 antigen fragments that are displayed by antigen-presenting cells in vivo. Accordingly, the present embodiments unexpectedly permit detection of SARS-CoV-2-specific immune response activity in a wide range of subjects having, suspected of having, or previously having been exposed to COVID-19 disease, which can be achieved by detecting a secondary in vitro response to the present compositions by T-cells from such subjects.

As also described below, for example, secondary in vitro T-cell immune responses were readily detected following stimulation by the herein disclosed CD8+ T-cell epitope containing SARS-CoV-2 spike protein oligopeptides (e.g., SEQ ID NOS: 1-20 and 39). Moreover, in certain embodiments such responses could unexpectedly be enhanced by inclusion of the herein disclosed CD4+ T-cell epitope containing SARS-CoV-2 spike protein receptor binding domain (RBD) oligopeptides comprising the amino acid sequences set forth in SEQ ID NOS: 22-36 in incubation admixtures of the herein described oligopeptides with samples containing T-cells from confirmed COVID-19 subjects.

Hence, according to non-limiting theory, the present embodiments permit rapid and sensitive detection of SARS-CoV-2-specific class I-restricted (e.g., CD8+) T-cells in a sample regardless of the stage of COVID-19 disease of the subject from whom the T-cells have been obtained. According to certain embodiments as described herein, the present peptide or oligopeptide compositions possess properties that are markedly different from any previously described SARS-CoV-2 peptides by providing unprecedented capability and enhanced sensitivity for the detection of SARS-CoV-2-specific CD8+ T-cell immune responsiveness.

Accordingly and as disclosed herein, certain embodiments provide an in vitro SARS-CoV-2-specific functional immunological test that detects an immune response indicator (e.g., interferon-γ (IFNγ)) produced by CD8+ T-cells that have been activated by exposure to specific regions within SARS-CoV-2 spike proteins. Specific T-cell reactive epitopes are described herein, and are believed, according to non-limiting theory, to find uses in the present compositions and methods as a consequence of the recognition by T cells of MHC class I-binding antigenic peptide epitopes.

### SARS-Cov-2 Spike CD8+ T-Cell Epitope-Containing Peptides.

In certain embodiments the present disclosure provides a composition for diagnosis or prognosis of coronavirus disease 2019 (Covid-19), or for detecting an antigen-specific T cell-mediated immune response to severe acute respiratory syndrome coronavirus 2 (SARS-CoV-2), comprising 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 and 39, preferably SEQ ID NOS: 1-20 and 39:

SEQ ID NO: 1
YPDKVFRSSVLHST,
SEQ ID NO: 2
VLHSTQDLFLPFF,
SEQ ID NO: 3
KSWMESEFRVY,
SEQ ID NO: 4
RVYSSANNCTFEY,
SEQ ID NO: 5
EFVFKNIDGYFK,
SEQ ID NO: 6
YYVGYLQPRTFLLKY,
SEQ ID NO: 7
EVFNATRFASVYAW,
SEQ ID NO: 8
RISNCVADYSVLYN,
SEQ ID NO: 9
YSVLYNSASFTFKCY,
SEQ ID NO: 10
CFTNVYADSFV,
SEQ ID NO: 11
LYRLFRKSNLKPF,
SEQ ID NO: 12
YQPYRVVVLSFEL,
SEQ ID NO: 13
WRVYSTGSNVFQ,
SEQ ID NO: 14
TNSPRRARSVASQSI,
SEQ ID NO: 15
RSVASQSIIAYTMSL,
SEQ ID NO: 16
MTKTSVDCTMY,
SEQ ID NO: 17
PLLTDEMIAQYTSALL,
SEQ ID NO: 18
AALQIPFAMQMAYRF,
SEQ ID NO: 19
RAAEIRASANLAATKM,
SEQ ID NO: 20
KYEQYIKWPWYIWLGFI,
SEQ ID NO: 21
YIWLGFIAGLIAIVM,
and
SEQ ID NO: 39
YHLMSFPQSAPH,

or one or more variants thereof having at least 80% amino acid sequence identity to the amino acid sequences set forth in SEQ ID NOS:1-21 and 39, preferably SEQ ID NOS: 1-20 and 39.

In certain contemplated embodiments, a first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope may comprise, for example, at least: (1) the amino acid sequences set forth in SEQ ID NOS: 1, 3, and 5 or one or more variants having at least 80% amino acid sequence identity thereto; (2) the amino acid sequences set forth in SEQ ID NOS: 2, 10, and 12 or one or more variants having at least 80% amino acid sequence identity thereto; (3) the amino acid sequences set forth in SEQ ID NOS: 3, 6, and 11 or one or more variants having at least 80% amino acid sequence identity thereto; or (4) the amino acid sequences set forth in SEQ ID NOS: 4, 19, and 20 or one or more variants having at least 80% amino acid sequence identity thereto. Accordingly it will be appreciated that in certain embodiments provided herein the first peptide composition need not comprise all 21 of the herein disclosed SARS-CoV-2 Spike protein S1 or S2 region CD8+ T-cell epitope peptides of SEQ ID NOS: 1-20 and 39, or of SEQ ID NOS: 1-21.

In certain other contemplated embodiments, the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope may comprise, for example at least: (1) the amino acid sequences set forth in SEQ ID NOS: 1, 3, and 5 or one or more variants having at least 80% amino acid sequence identity thereto; (2) the amino acid sequences set forth in SEQ ID NOS: 2, 4, and 7 or one or more variants having at least 80% amino acid sequence identity thereto; (3) the amino acid sequences set forth in SEQ ID NOS: 8, 10, and 12 or one or more variants having at least 80% amino acid sequence identity thereto; (4) the amino acid sequences set forth in SEQ ID NOS: 9, 14, and 15 or one or more variants having at least 80% amino acid sequence identity thereto; (5) the amino acid sequences set forth in SEQ ID NOS: 6, 11, and 18 or one or more variants having at least 80% amino acid sequence identity thereto; (6) the amino acid sequences set forth in SEQ ID NOS: 13, 16, and 39 or one or more variants having at least 80% amino acid sequence identity thereto; or (7) the amino acid sequences set forth in SEQ ID NOS: 17, 19, and 20 or one or more variants having at least 80% amino acid sequence identity thereto. Accordingly it will be appreciated that in certain embodiments provided herein the first peptide composition need not comprise all 21 of the herein disclosed SARS-CoV-2 Spike protein S1 or S2 region CD8+ T-cell epitope peptides of SEQ ID NOS: 1-20 and 39, or of SEQ ID NOS: 1-21.

In certain embodiments a composition for diagnosis or prognosis of coronavirus disease 2019 (Covid-19), or for detecting an antigen-specific T cell-mediated immune response to severe acute respiratory syndrome coronavirus 2 (SARS-CoV-2) comprises, and in certain embodiments the above described composition further comprises, 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36:

SEQ ID NO: 22
RVQPTESIVRFPNITNLCPFGEVEN,
SEQ ID NO: 23
NLCPFGEVFNATRFASVYAWNRKRI,
SEQ ID NO: 24
SVYAWNRKRISNCVADYSVLYNSAS,
SEQ ID NO: 25
DYSVLYNSASFSTFKCYGVSPTKLN,
SEQ ID NO: 26
CYGVSPTKLNDLCFTNVYADSFVIR,
SEQ ID NO: 27
NVYADSFVIRGDEVRQIAPGQTGKI,
SEQ ID NO: 28
RQIAPGQTGKIADYNYKLPDDFTGC,
SEQ ID NO: 29
YKLPDDFTGCVIAWNSNNLDSKVGG,
SEQ ID NO: 30
SNNLDSKVGGNYNYLYRLFRKSNLK,
SEQ ID NO: 31
YRLFRKSNLKPFERDISTEIYQAGS,
SEQ ID NO: 32
ISTEIYQAGSTPCNGVEGFNCYFPL,
SEQ ID NO: 33
VEGFNCYFPLQSYGFQPTNGVGYQP,
SEQ ID NO: 34
FQPTNGVGYQPYRVVVLSFELLHAP,
SEQ ID NO: 35
VLSFELLHAPATVCGPKKSTNLVKN,
and
SEQ ID NO: 36
PKKSTNLVKNKCVNF,

or one or more variants thereof having at least 80% amino acid sequence identity to the amino acid sequences set forth in SEQ ID NOS:22-36.

A SARS-CoV-2 spike protein peptide or oligopeptide that comprises a CD8+ T-cell epitope for use in certain embodiments contemplated herein may comprise the amino acid sequence set forth in any one of SEQ ID NOS:1-21 and 39, preferably SEQ ID NOS: 1-20 and 39, and may in certain other embodiments comprise a SARS-CoV-2 spike protein CD8+ T-cell epitope-containing peptide variant comprising a peptide having an amino acid sequence that is at least 80%, 81%7 82%7 83%7 84%7 85%, 86%, 87%7 88%, 89%, 90%, 91%, 92%, 93%, 94%, 95%, 96%, 97%, 98% or 99% identical to at least one of such peptides of SEQ ID NOS:1-21 and 39 and that is capable of being specifically recognized by a T-cell that is reactive with at least one pathogenic SARS-CoV-2 strain, preferably a strain that is pathogenic in humans. Amino acid sequences of SARS-CoV-2 Spike protein S1 or S2 region CD8+ T-cell epitope-containing peptides of SEQ ID NOS: 1-21 and 39 are set forth using the well known single-letter amino acid code in the Examples below in Table 1, and also elsewhere herein.

SARS-CoV-2 Spike protein S1 or S2 region CD8+ T-cell epitope-containing peptide variants of any of the peptides set forth as SEQ ID NOS: 1-21 and 39, preferably SEQ ID NOS: 1-20 and 39, may contain one or more amino acid substitutions, additions, deletions, and/or insertions relative to the T-cell epitope-containing peptide sequence set forth in SEQ ID NOS: 1-21 and 39 (e.g., wildtype, or predominant or naturally occurring allelic forms). Variants preferably exhibit at least about 80%, 81%7 82%, 83%, 84%, 85%, 86%, 87%, 88%, or 89% amino acid sequence identity and more preferably at least about 90%, 91%, 92%, 93%, 94%, 95%, 96%, 97%, 98% or 99% amino acid sequence identity to a corresponding portion of a native SARS-CoV-2 Spike protein S1 or S2 region CD8+ T-cell epitope-containing polypeptide sequence region. The percent identity may be readily determined by comparing sequences of the peptide variants with the corresponding portion of a full-length polypeptide, where corresponding portions can be readily identified according to established methods, for example, by aligning sequence regions that exhibit a high degree of sequence identity or sequence homology, optionally allowing for short sequence gaps as may arise due to insertions or deletions, or for conservative substitutions, or for short mismatched regions, or the like. Some techniques for sequence comparison include using computer algorithms well known to those having ordinary skill in the art, such as Align or the BLAST algorithm (Altschul, J. Mol. Biol. 219:555-565, 1991; Henikoff and Henikoff, PNAS USA 89:10915-10919, 1992), which is available at the NCBI website (see [online] Internet:<URL: http://www/ncbi.nlm.nih.gov/cgi-bin/BLAST). Default or custom parameters may be used.

Furthermore, computer algorithms are available in the art that enable the skilled artisan to predict the three-dimensional structure of a protein or peptide, in order to ascertain functional variants of a particular polypeptide. For instance, variants can be identified wherein all or a portion of the three-dimensional structure is not substantially altered by one or more modification, substitution, addition, deletion and/or insertion. (See, for example, DeepMind AlphaFold (London, UK), 2020 (30 November) Nature d41586-020-03348-4; Seemayer et al., 2014 Bioinformat. 30:3128; Raman et al., 2010 ScienceExpress 4 Feb. 2010 10.1126/science.1183649; Gribenko et al., 2009 Proc. Nat. Acad. Sci. USA 106:2601; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Baker, 2014 Biochem. Soc. Trans. 42:225; Correia et al., 2014 Nature 507:201; King et al., 2014 Proc. Nat. Acad. Sci. USA 111:8577; Roche et al., 2012 PLoS One 7(5):e38219; Zhang et al., 2013 Meths. Enzymol. 523:21; Khoury et al., 2014 Trends Biotechnol. 32:99; O'Meara et al., 2015 J. Chem. Theory Comput. 11:609; Park et al., 2015 Structure 23:1123; Bale et al., 2015 Protein Sci. doi:10.1002/pro.2748 Epub PMID 26174163; Park et al., 2015 Proteins doi: 10.1002/prot.24862 Epub PMID 26205421; Lin et al., 2015 Proc. Nat. Acad. Sci. USA pii:201509508 Epub PMID 26396255). In this way, one of skill in the art can readily determine whether a particular SARS-CoV-2 CD8+ T-cell epitope-containing peptide variant, or a functional fragment thereof, retains sufficient epitope structure so as to be capable of being specifically recognized by a T-cell that is reactive with at least one pathogenic SARS-CoV-2 strain.

A SARS-CoV-2 Spike protein subunit 51 (amino acids 13-685 of SEQ ID NO: 37) or subunit S2 (amino acids 686-1273 of SEQ ID NO: 37) region CD8+ T-cell epitope-containing peptide may be a peptide of at least 5, 6, 7, 8, 9, 10, 11, 12, 13, 14, 15, 16, 17, 18, 19, or 20 contiguous amino acids and in certain embodiments may typically be not more than 100, 95, 90, 85, 80, 75, 70, 65, 60, 55, or 50 amino acids in length. In certain preferred embodiments the SARS-CoV-2 Spike protein S1 or S2 region CD8+ T-cell epitope-containing peptide may be a peptide of 8, 9, 10, 11, 12, 13, 14, 15, 16, 17, 18, 19, or 20 amino acids, or in certain embodiments, a peptide of 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous amino acids in length.

It is understood that a T-cell epitope refers to a structural region of an antigen that can be specifically recognized by a T-cell receptor for antigen (“T-cell receptor”), typically in the context of an appropriate major histocompatibility complex (MHC) class I or class II molecule that presents the epitope to the T-cell receptor. T-cell epitope-containing peptides of about 8-13 amino acids in length are typically presented to CD8+ T-cell receptors by class I MHC molecules (e.g., human leukocyte antigens HLA-A, -B,-C); T-cell epitope-containing peptides of about 15-25 amino acids in length are typically presented to CD4+ T-cell receptors by class II MHC molecules (e.g., human leukocyte antigens HLA-DP, -DQ,-DR). T-cell receptors are not absolute in their specificity but are instead regarded as promiscuous; that is to say, a given T-cell receptor may be capable of specifically recognizing a particular T-cell epitope structure and also a range of closely related epitope structures. Specific recognition of an appropriately presented T-cell epitope by a T-cell may be detectable as stimulation of the generation of a T-cell immune response indicator such as those described herein (e.g., cytokine release by T-cells, such as IFN-γ release). Hence a SARS-CoV-2 Spike protein S1 or S2 region CD8+ T-cell epitope-containing peptide may refer to a peptide antigen that is capable, in a secondary in vitro immune response, of stimulating a T-cell that has been primed (e.g., activated) to recognize a SARS CoV-2 spike protein antigen, including situations where the SARS-CoV-2 Spike protein S1 or S2 region CD8+ T-cell epitope-containing peptide is not identical to the SARS-CoV-2 antigen with which the T-cell may have been primed in vivo.

Methodologies for the design, production and testing of SARS-CoV-2 Spike protein S1 or S2 region CD8+ T-cell epitope-containing peptides and variants functional fragments thereof as provided herein are all available by minor modification to existing knowledge in the art, for example, using conventional methods of virology, immunology, microbiology, molecular biology and recombinant DNA techniques, which are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The terms “polypeptide”, “protein”, “peptide”, and “oligopeptide” are used interchangeably and mean a polymer of amino acids in peptide linkage that need not limited to any particular length. The term does not exclude modifications such as myristylation, sulfation, glycosylation, phosphorylation, formylation, and addition or deletion of signal sequences. The term “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains. The terms “peptide,” “oligopeptide”, “polypeptide” and “protein” specifically encompass the peptides of the present disclosure, or sequences that have deletions from, additions to, and/or substitutions of one or more amino acid of a herein described peptide.

The term “isolated” means that the material is removed from its original environment (e.g., the natural environment if it is naturally occurring). For example, a naturally occurring polypeptide or nucleic acid present in a living animal is not isolated, but the same polypeptide or nucleic acid, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

The terms “isolated protein”, “isolated polypeptide” and “isolated peptide” referred to herein means that a subject protein, peptide or polypeptide (1) is free of at least some other proteins, peptides or polypeptides with which it would typically be found in nature, (2) is essentially free of other proteins, peptides or polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein or polypeptide with which the isolated protein, isolated peptide or isolated polypeptide may be associated in nature, (6) is operably associated (by covalent or noncovalent interaction) with a polypeptide with which it is not associated in nature, or (7) does not occur in nature. Such an isolated protein, peptide or polypeptide can be encoded by genomic DNA, cDNA, mRNA or other RNA, or may be of synthetic origin according to any of a number of well known chemistries for artificial peptide and protein synthesis, or any combination thereof. In certain embodiments, the isolated protein, peptide or polypeptide is substantially free from proteins or polypeptides or other contaminants that are found in its natural environment that would interfere with its use (therapeutic, diagnostic, prophylactic, research or otherwise).

A “peptide fragment” or “polypeptide fragment” refers to a peptide or polypeptide, which can be monomeric or multimeric, that has an amino-terminal deletion, a carboxyl-terminal deletion, and/or an internal deletion or substitution of a naturally-occurring or recombinantly-produced polypeptide. As used herein, “contiguous amino acids” refers to covalently linked amino acids corresponding to an uninterrupted linear portion of a disclosed amino acid sequence. In certain embodiments, a polypeptide fragment can comprise an amino acid chain at least 5 to about 100 amino acids long. It will be appreciated that in certain embodiments, fragments are at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 amino acids long.

Certain preferred embodiments contemplate wholly artificial chemical synthesis of the herein described peptides (e.g., a SARS-CoV-2 Spike protein S1 or S2 region CD8+ T-cell epitope-containing peptide) according to any of a number of established methodologies, such as those described in Amino Acid and Peptide Synthesis (Jones, J., 2002 Oxford Univ. Press USA, New York), Ramakers et al. (2014 Chem. Soc. Rev. 43:2743), Verzele et al. (2013 Chembiochem. 14:1032), Chandrudu et al. (2013 Molecules 18:4373), and/or Made et al. (2004 Beilstein J. Org. Chem. 10:1197). For example, manual or preferably automated solid-phase peptide synthesis based on the Merrifield method or other solid-phase peptide synthetic techniques and subsequent improvements (e.g., Merrifield, 1963 J. Am. Chem. Soc. 85:2149; Mitchell et al., 1978 J. Org. Chem. 43:2485; Albericio, F. (2000). Solid-Phase Synthesis: A Practical Guide (1 ed.). Boca Raton: CRC Press; Nilsson et al., 2005 Annu. Rev. Biophys. Biomol. Struct. 34; Schnolzer et al., Int. J. Peptide Res. Therap. 13 (1-2): 31; Li et al. 2013 Molecules 18:9797) are routine in the peptide synthesis art and may be employed to chemically synthesize the herein described peptides.

A polypeptide or peptide may comprise a signal (or leader) sequence at the N-terminal end of the protein, which co-translationally or post-translationally directs transfer of the protein. The polypeptide or peptide may also be fused in-frame or conjugated to a linker or other sequence for ease of synthesis, purification or identification of the polypeptide (e.g., poly-His), or to enhance binding of the polypeptide or peptide to a solid support. Fusion domain polypeptides may be joined to the polypeptide or peptide at the N-terminus and/or at the C-terminus, and may include as non-limiting examples, immunoglobulin-derived sequences such as Ig constant region sequences or portions thereof, affinity tags such as His tag (e.g., hexahistidine or other polyhistidine), FLAG™ or myc or other peptide affinity tags, detectable polypeptide moieties such as green fluorescent protein (GFP) or variants thereof (e.g., yellow fluorescent protein (YFP), blue fluorescent protein (BFP), other aequorins or derivatives thereof, etc.) or other detectable polypeptide fusion domains, enzymes or portions thereof such as glutathione-S-transferase (GST) or other known enzymatic detection and/or reporter fusion domains, and the like, as will be familiar to the skilled artisan.

Systems for recombinant expression of peptides, polypeptides and proteins are known in the art and may in certain embodiments be used to produce the herein described peptides. For example, certain bacterial expression systems such as E. coli recombinant protein expression systems yield polypeptide products having N-terminal formylated methionine. In some situations a recombinantly produced peptide may therefore comprise an N-terminal methionine residue (which may be unmodified methionine or formylmethionine or another methionine analog, variant, mimetic or derivative as provided herein), sometimes referred to as initiator methionine, immediately preceding the desired peptide sequence (e.g., the SARS-CoV-2 Spike protein S1 or S2 region CD8+ T-cell epitope-containing peptide). Thus also contemplated are embodiments in which one or more of the herein described SARS-CoV-2 Spike protein S1 or S2 region CD8+ T-cell epitope-containing peptides are generated containing N-terminal methionine (e.g., as methionine or N-formylmethionine) and may be recombinantly expressed according to art-accepted practices in a host cell that also expresses methionine aminopeptidase (MAP), an enzyme that is capable of cleaving the N-terminal methionine to remove it from the nascent polypeptide product. See, e.g., Natarajan et al., 2011 PLoS ONE 6(5): e20176; Shen et al., 1993 Proc. Natl. Acad. Sci. USA 90:8108; Shen et al., 1997 Prot. Eng. 10:1085. Alternatively, the MAP enzyme itself may be produced recombinantly (e.g., Tsunasawa et al., 1997 J. Biochem. 122:843; Bradshaw et al., 1998 Trends Bloch. Sci. 23:263; Ben-Bassat et al., 1987 J. Bacteriol. 169:751) or obtained commercially (Sigma-Aldrich, St. Louis, MO, e.g., catalog number M6435) and used to remove N-terminal methionine from the present peptides post-synthesis.

According to certain preferred embodiments a SARS-CoV-2 Spike protein S1 or S2 region CD8+ T-cell epitope-containing peptide may comprise a peptide, oligopeptide, polypeptide or peptidomimetic that includes, or that shares close sequence identity to or structural features with, a polypeptide of at least 5 and no more than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7 or 6 amino acids, comprising the amino acid sequence set forth in any one of SEQ ID NOS:1-21 and 39, preferably SEQ ID NOS: 1-20 and 39, wherein the peptide in which the SARS-CoV-2 Spike protein S1 or S2 region CD8+ T-cell epitope is present is capable of being specifically recognized by a T-cell that is reactive with at least one pathogenic SARS-CoV-2 strain, preferably one that is pathogenic in humans. Assay methods for determining such T-cell reactivity are described herein and are also known generally in the art, except for the identities of SARS-CoV-2 Spike protein S1 or S2 region CD8+ T-cell epitope-containing proteins and peptides which are disclosed herein for the first time.

As generally referred to in the art, and as used herein, sequence identity and sequence homology may be used interchangeably and generally refer to the percentage of nucleotides or amino acid residues in a candidate sequence that are identical with, respectively, the nucleotides or amino acid residues in a reference polynucleotide or polypeptide sequence, after aligning the sequences and introducing gaps, if necessary, to achieve the maximum percent sequence identity, and optionally not considering any conservative substitutions as part of the sequence identity. In certain embodiments, a variant of a peptide such as a herein disclosed SARS-CoV-2 Spike protein S1 or S2 region CD8+ T-cell epitope-containing peptide (e.g., a peptide according to one of SEQ ID NOS:1-21 and 39, preferably SEQ ID NOS: 1-20 and 39) shares at least about 80%, at least about 85%, at least about 90%, 91%, 92%, 93% or 94%, or at least about 95%, 96%, 97%, 98%, or 99% of the amino acid residues (or of the nucleotides in a polynucleotide encoding such a peptide) with the sequence of the peptide of any one of SEQ ID NOS:1-21 and 39. Such sequence identity may be determined according to well known sequence analysis algorithms, as also noted above, and including those available from the University of Wisconsin Genetics Computer Group (Madison, WI), such as FASTA, Gap, Bestfit, BLAST, or others.

“Natural or non-natural amino acid” includes any of the common naturally occurring amino acids which serve as building blocks for the biosynthesis of peptides, polypeptides and proteins (e.g., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), tyrosine(Y)) and also includes modified, derivatized, enantiomeric, rare and/or unusual amino acids, whether naturally occurring or synthetic, for instance, N-formylmethionine, hydroxyproline, hydroxylysine, desmosine, isodesmosine, ε-N-methyllysine, ε-N-trimethyllysine, methylhistidine, dehydrobutyrine, dehydroalanine, α-aminobutyric acid, β-alanine, γ-amino butyric acid, homocysteine, homoserine, citrulline, ornithine and other amino acids that may be isolated from a natural source and/or that may be chemically synthesized, for instance, as may be found in Proteins, Peptides and Amino Acids Sourcebook (White, J. S. and White, D. C., 2002 Humana Press, Totowa, NJ) or in Amino Acid and Peptide Synthesis (Jones, J., 2002 Oxford Univ. Press USA, New York) or in Unnatural Amino Acids, ChemFiles Vol. 1, No. 5 (2001 Fluka Chemie GmbH; Sigma-Aldrich, St. Louis, MO) or in Unnatural Amino Acids II, ChemFiles Vol. 2, No. 4 (2002 Fluka Chemie GmbH; Sigma-Aldrich, St. Louis, MO). Additional descriptions of natural and/or non-natural amino acids may be found, for example, in Kotha, 2003 Acc. Chem. Res. 36:342; Maruoka et al., 2004 Proc. Nat. Acad. Sci. USA 101:5824; Lundquist et al., 2001 Org. Lett. 3:781; Tang et al., 2002 J. Org. Chem. 67:7819; Rothman et al., 2003 J. Org. Chem. 68:6795; Krebs et al., 2004 Chemistry 10:544; Goodman et al., 2001 BiopoEEEErs 60:229; Sabat et al., 2000 Org. Lett. 2:1089; Fu et al., 2001 J. Org. Chem. 66:7118; and Hruby et al., 1994 Meths. Mol. Biol. 35:249. The standard three-letter abbreviations and one-letter symbols are used herein to designate natural and non-natural amino acids.

Other non-natural amino acids or amino acid analogues are known in the art and include, but are not limited to, non-natural L or D derivatives (such as D-amino acids present in peptides and/or peptidomimetics such as those presented above and elsewhere herein), fluorescent labeled amino acids, as well as specific examples including O-methyl-L-tyrosine, an L-3-(2-naphthyl)alanine, a 3-methyl-phenylalanine, 3-idio-tyrosine, O-propargyl-tyrosine, homoglutamine, an O-4-allyl-L-tyrosine, a 4-propyl-L-tyrosine, a 3-nitro-L-tyrosine, a tri-O-acetyl-GIcNAcβ-serine, an L-Dopa, a fluorinated phenylalanine, an isopropyl-L-phenylalanine, a ρ-azido-L-phenylalanine, a ρ-acyl-L-phenylalanine, a ρ-acetyl-L-phenylalanine, an m-acetyl-L-phenylalanine, selenomethionine, telluromethionine, selenocysteine, an alkyne phenylalanine, an O-allyl-L-tyrosine, an O-(2-propynyl)-L-tyrosine, a ρ-ethylthiocarbonyl-L-phenylalanine, a ρ-(3-oxobutanoyl)-L-phenylalanine, a ρ-benzoyl-L-phenylalanine, an L-phosphoserine, a phosphonoserine, a phosphonotyrosine, homoproparglyglycine, azidohomoalanine, a ρ-iodo-phenylalanine, a ρ-bromo-L-phenylalanine, dihydroxy-phenylalanine, dihydroxyl-L-phenylalanine, a ρ-nitro-L-phenylalanine, an m-methoxy-L-phenylalanine, a ρ-iodo-phenylalanine, a ρ-bromophenylalanine, a ρ-amino-L-phenylalanine, and an isopropyl-L-phenylalanine, trifluoroleucine, norleucine (“Nle”), D-norleucine (“dNle” or “D-Nle”), 5-fluoro-tryptophan, para-halo-phenylalanine, homo-phenylalanine (“homo-Phe”), seleno-methionine, ethionine, S-nitroso-homocysteine, thia-proline, 3-thienyl-alanine, homo-allyl-glycine, trifluoroisoleucine, trans and cis-2-amino-4-hexenoic acid, 2-butynyl-glycine, allyl-glycine, para-azido-phenylalanine, para-cyano-phenylalanine, para-ethynyl-phenylalanine, hexafluoroleucine, 1,2,4-triazole-3-alanine, 2-fluoro-histidine, L-methyl histidine, 3-methyl-L-histidine, β-2-thienyl-L-alanine, β-(2-thiazolyl)-DL-alanine, homoproparglyglycine (HPG) and azidohomoalanine (AHA) and the like.

In certain embodiments a natural or non-natural amino acid may be present that comprises an aromatic side chain, as found, for example, in phenylalanine or tryptophan or analogues thereof including in other natural or non-natural amino acids based on the structures of which the skilled person will readily recognize when an aromatic ring system is present, typically in the form of an aromatic monocyclic or multicyclic hydrocarbon ring system consisting only of hydrogen and carbon and containing from 6 to 19 carbon atoms, where the ring system may be partially or fully saturated, and which may be present as a group that includes, but need not be limited to, groups such as fluorenyl, phenyl and naphthyl.

In certain embodiments a natural or non-natural amino acid may be present that comprises a hydrophobic side chain as found, for example, in alanine, valine, isoleucine, leucine, proline, phenylalanine, tryptophan or methionine or analogues thereof including in other natural or non-natural amino acids based on the structures of which the skilled person will readily recognize when a hydrophobic side chain (e.g., typically one that is non-polar when in a physiological milieu) is present. In certain embodiments a natural or non-natural amino acid may be present that comprises a basic side chain as found, for example, in lysine, arginine or histidine or analogues thereof including in other natural or non-natural amino acids based on the structures of which the skilled person will readily recognize when a basic (e.g., typically polar and having a positive charge when in a physiological milieu) is present.

Peptides disclosed herein may in certain embodiments include L- and/or D-amino acids so long as the biological activity of the peptide is maintained (e.g., the SARS-CoV-2 Spike protein S1 or S2 region CD8+ T-cell epitope-containing peptide is capable of being recognized in a secondary in vitro immune response by T-cells from a subject infected with a SARS-CoV-2 strain that is associated with COVID-19 disease, as evidenced by stimulation of the generation of a T-cell immune response indicator as described herein). The peptides also may comprise in certain embodiments any of a variety of known natural and artificial post-translational or post-synthetic covalent chemical modifications by reactions that may include glycosylation (e.g., N-linked oligosaccharide addition at asparagine residues, O-linked oligosaccharide addition at serine or threonine residues, glycation, or the like), fatty acylation, acetylation, formylation, PEGylation, and phosphorylation. Peptides herein disclosed may further include analogs, alleles and allelic variants which may contain amino acid deletions, or additions or substitutions of one or more amino acid residues with other naturally occurring amino acid residues or non-natural amino acid residues.

Peptide and non-peptide analogs may be referred to as peptide mimetics or peptidomimetics, and are known in the pharmaceutical industry (Fauchere, J. Adv. Drug Res. 15:29 (1986); Evans et al. J. Med. Chem. 30: 1229 (1987)). These compounds may contain one or more non-natural amino acid residue(s), one or more chemical modification moieties (for example, glycosylation, pegylation, fluorescence, radioactivity, or other moiety), and/or one or more non-natural peptide bond(s) (for example, a reduced peptide bond: —CH₂—NH₂—). Peptidomimetics may be developed by a variety of methods, including by computerized molecular modeling, random or site-directed mutagenesis, PCR-based strategies, chemical mutagenesis, and others.

As also described above, certain embodiments also relate to peptidomimetics, or “artificial” polypeptides. Such polypeptides may contain one or more amino acid insertions, deletions or substitutions, one or more altered or artificial peptide bond, one or more chemical moiety (such as polyethylene glycol, glycosylation, a detectable label, or other moiety), and/or one or more non-natural amino acid. Synthesis of peptidomimetics is well known in the art and may include altering naturally occurring proteins or polypeptides by chemical mutagenesis, single or multi-site-directed mutagenesis, PCR shuffling, use of altered aminoacyl tRNA or aminoacyl tRNA synthetase molecules, the use of “stop” codons such as amber suppressors, the use of four or five base-pair codons, or other means.

Any combination of one or more of the CD8+ T-cell epitope-containing peptides of Table 1 may be employed in certain presently contemplated embodiments of the herein disclosed compositions and methods, and in certain preferred embodiments each of the peptides of SEQ ID NOS: 1-20 and 39, or of SEQ ID NOS: 1-21, or of SEQ ID NOS: 1-21 and 39, will be present. In certain further embodiments any combination of one or more of the CD4+ T-cell epitope-containing peptides set forth in SEQ ID NOS: 22-36 may also be present, and in certain preferred embodiments each of the peptides of SEQ ID NOS: 22-36 will be present.

The amounts of the several peptides relative to one another, and the absolute amounts of one or more of said peptides, may be varied according to the assay design and particular technique in which the composition is to be used, as will be familiar to the skilled artisan according to particular immunochemical, immunological and/or biochemical methodologies. By way of illustration and not limitation, in certain embodiments the composition may comprise at least about one nanogram of each peptide and not more than about 100 nanograms of each peptide; in certain embodiments the composition may comprise at least about 100, 200, 300, or 400 nanograms and not more than about 500 nanograms of each peptide; in certain embodiments the composition may comprise at least about 500, 600, 700, 800, or 900 nanograms and not more than about 1000 nanograms of each peptide; in certain embodiments the composition may comprise at least about 1.0, 1.1, 1.2, 1.3, 1.4, 1.5, 1.6, 1.7, 1.8, or 1.9 micrograms and not more than about 2 micrograms of each peptide, and in certain embodiments the composition may comprise at least about 1, 2, 3, 4, 5, 6, 7, 8, or 9 micrograms and not more than about 10 micrograms of each peptide.

### Methods for Diagnosis or Prognosis of Covid-19 Disease and for Detection of CD8+ SARS-Cov-2-Reactive T Cells

As disclosed herein there are provided, in certain embodiments, compositions and methods for detecting COVID-19 disease in a subject, or for determining prior exposure of the immune system in a subject to SARS-CoV-2, based in part on the discovery that a peptide composition containing at least one herein described SARS-CoV-2 Spike protein S1 or S2 region CD8+ T-cell epitope-containing peptide (e.g., Table 1, SEQ ID NOS: 1-21 and 39), and in some embodiments 2, 3, 4, 5, 6, 7, 8, 9, 10, 11, 12, 13, 14, 15, 16, 17, 18, 19, 20, or all 21 of the oligopeptides having the amino acid sequences set forth in SEQ ID NOS: 1-20 and 39, or variants having at least 80% amino acid sequence identity to such oligopeptides, provides a composition with which, by way of non-limiting theory, T-cells from substantially all COVID-19 patients will react in a secondary in vitro immune response, by generating a T-cell immune response indicator (e.g., interferon-γ release) as provided herein.

These and related embodiments thus permit detection of SARS-CoV-2-specific cell-mediated immunological reactivity in a subject at a point in time that may be long after the time at which SARS-CoV-2-specific antibodies can be detected in a biological sample obtained from the subject.

In certain embodiments, the present disclosure contemplates assessment of a COVID-19 patient sample for CD8+ T cell-mediated immunoresponsiveness in vitro to one or more of the herein described CD8+ T-cell epitope-containing SARS-CoV-2 spike protein oligopeptides (SEQ ID NOS: 1-21 and 39, or variants thereof, preferably SEQ ID NOS: 1-20 and 39, or variants thereof), optionally as may be enhanced by the inclusion in the in vitro assay of any one or more of the herein described CD4+ T-cell epitope-containing SARS-CoV-2 spike protein peptides (SEQ ID NOS: 22-36, or variants thereof). In these and related embodiments there is thus provided a method to monitor disease progression, remission, and/or relapse.

Accordingly, the present methods may be exploited to monitor the efficacy of a treatment for COVID-19 that is administered to a subject before and/or after sample collection, based on an altered (e.g., decreased or increased in a statistically significant manner, relative to an appropriate control) level in detectable antigen-specific T-cell responsiveness to one or more of the presently disclosed SARS-CoV-2 spike protein CD8+ T-cell epitope-containing peptides by circulating T-cells obtained from the subject. For example, in certain embodiments successful treatment (or even a successful immune response) to eradicate SARS-CoV-2 may be indicated by a decrease over time in the intensity or rapidity of a secondary in vitro T cell-mediated response to spike protein antigenic peptides by lymphocytes obtained at a series of timepoints post-treatment from a COVID-19 patient. By way of non-limiting theory, such a decline over time in the in vitro SARS-CoV-2 antigen-specific immunoreactivity in the patient sample may reflect a diminishing presence of SARS-CoV-2-specific T cells in a patient for whom immune protection against the virus may no longer be needed where there is no longer a viral antigen load to stimulate an immune response.

Conversely, in other embodiments it may be envisioned that the presently disclosed methods will permit demonstration, in serial samples collected over time, of a sustained anti-SARS-CoV-2 immunocompetence manifest as persistently detectable secondary in vitro antigen-specific CD8+ T-cell immunoreactivity against the herein described SARS-CoV-2 spike protein epitope-containing peptides. Such persistent immunoresponsiveness, according to non-limiting theory, may reflect the generation of long-lived memory T cells, for example by a SARS-CoV-2 infection or, alternatively, by a successful vaccination protocol. Further according to theory, the ability to detect a persistent SARS-CoV-2-specific presence in the T cell compartment may advantageously complement serological testing for SARS-CoV-2-specific antibodies, which would be expected to decline in abundance following a cleared viral infection (or a vaccination) and hence would not be useful for assessing potential susceptibility to reinfection.

Accordingly and in certain embodiments there is provided a method for detecting COVID-19 disease in a subject, or for monitoring efficacy of a treatment for COVID-19 disease in a subject, comprising (A) contacting in vitro (i) a first biological sample obtained at a first timepoint a subject known to have or suspected of being at risk for having COVID-19 disease, wherein the biological sample comprises T-cells and antigen-presenting cells, and (ii) a peptide composition for diagnosis or prognosis of COVID-19 disease, to obtain a first test incubation mixture; (B) incubating the first test incubation mixture under conditions and for a time sufficient for specific recognition by said T-cells of a SARS-CoV-2 spike protein CD8+ T-cell epitope that is present in said peptide composition to stimulate generation of a T-cell immune response indicator; and (C) detecting a first level of the T-cell immune response indicator in the first test incubation mixture, wherein presence of a COVID-19 infection in the subject is indicated by detection in (C) of said first level of the T-cell immune response indicator that is increased relative to a first control level of the T-cell immune response indicator obtained by incubating the first biological sample in a first control incubation without the peptide composition for diagnosis or prognosis of COVID-19 disease, and wherein the peptide composition for diagnosis or prognosis of COVID-19 disease comprises a peptide cocktail containing at least one SARS-CoV-2 spike protein CD8+ T-cell epitope-containing peptide comprising the amino acid sequence selected from SEQ ID NOS: 1-21 or more preferably SEQ ID NOS: 1-20 and 39, or a variant having at least 80% amino acid sequence identity to such peptide(s), for example:
(a) 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 or more preferably SEQ ID NOS: 1-20 and 39, or one or more variants thereof having at least 80% amino acid sequence identity to the amino acid sequences set forth in SEQ ID NOS:1-21 or more preferably SEQ ID NOS: 1-20 and 39, or
(b) a set of 21 isolated oligopeptides that comprise the amino acid sequences set forth in SEQ ID NOS: 1-21 or more preferably SEQ ID NOS: 1-20 and 39, or one or more variants thereof having at least 80% amino acid sequence identity to the amino acid sequences set forth in one or more of SEQ ID NOS:1-21 or more preferably SEQ ID NOS: 1-20 and 39,
and thereby detecting COVID-19 disease in the subject, or monitoring efficacy of the treatment or vaccine for COVID-19 disease in the subject.

In certain further embodiments, the peptide composition for diagnosis or prognosis of COVID-19 disease additionally comprises any one or more (e.g., 1, 2, 3, 4, 5, 6, 7, 8, 9, 10, 11, 12, 13, 14, or all 15) of the 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

In certain preferred embodiments, the first timepoint is prior to administration to the subject of treatment for COVID-19 disease.

In certain further embodiments the method further comprises (D) contacting in vitro (i) a second biological sample obtained from the subject at a second timepoint that is later than the first timepoint and is after administration to the subject of treatment for COVID-19 disease, wherein the second biological sample comprises T-cells and antigen-presenting cells, and (ii) the peptide composition for diagnosis or prognosis of COVID-19 disease, to obtain a second test incubation mixture; (E) incubating the second test incubation mixture under conditions and for a time sufficient for specific recognition by said T-cells of a SARS-CoV-2 CD8+ T-cell epitope that is present in said peptide composition to stimulate generation of a T-cell immune response indicator; and (F) detecting a second level of the T-cell immune response indicator in the second test incubation mixture, wherein presence of a SARS-CoV-2 infection in the subject is indicated by detection in (F) of said second level of the T-cell immune response indicator that is increased relative to a second control level of the T-cell immune response indicator obtained by incubating the second biological sample in a second control incubation without the peptide composition for diagnosis or prognosis of COVID-19 disease, and wherein efficacy of the treatment for COVID-19 disease is indicated by detection in (F) of said second level of the T-cell immune response indicator that is decreased relative to the first level of the T-cell immune response indicator that is detected in (C).

In these and certain related embodiments, it will therefore be recognized that the first timepoint is prior to administration to the subject of a given treatment for COVID-19 disease, whilst the second or subsequent timepoints may be after administration to the subject of the given treatment for COVID-19, such that efficacy of the treatment may be determined, as reflected, for example, by a decrease (e.g., a statistically significant reduction relative to an appropriate control) in the second level of the T-cell immune response indicator that is detected. By way of non-limiting theory, such a result would signify that at the second or subsequent timepoint, the second biological sample contains lower levels of SARS-CoV-2-specific T-cell reactivity when assayed for stimulation of generation of a T-cell immune response indicator relative to the first timepoint, as a consequence of negatively-regulated and/or absent T-cell reactivity in the second sample due to substantial clearance of the SARS-CoV-2 infection in the subject following the COVID-19 treatment.

In this manner it will be appreciated that the severity of a SARS-CoV-2 infection associated with COVID-19 may be monitored over various time periods, such as over the course of one or a plurality of second timepoints, to assess disease progression as reflected by T-cell immune response indicator level as an apparent indicator of SARS-CoV-2 viral load in the subject, and also to assess the efficacy of one or more treatments for COVID-19. It may in certain cases be desirable to repeatedly test a plurality of biological samples obtained from a subject over a succession of second and subsequent timepoints in order to monitor the SARS-CoV-2-specific T-cell activity in the subject. Accordingly, in certain embodiments the presently described steps of contacting, incubating, and detecting may be repeated any number of times in situations where it may be desirable to monitor COVID-19 in a subject over an extended time period, for example over a plurality of second timepoints such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more second timepoints, which may be separated from one another by variable intervals that may be intervals of several days, weeks, months, or years.

As understood by a person skilled in the medical art, the terms, “treat” and “treatment,” refer to medical management of a disease, disorder, or condition of a subject (i.e., patient, host, who may be a human or non-human animal) (see, e.g., Stedman's Medical Dictionary).

In certain embodiments of the herein described methods, the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope may, for example, comprise at least: (1) the amino acid sequences set forth in SEQ ID NOS: 1, 3, and 5 or one or more variants having at least 80% amino acid sequence identity thereto; (2) the amino acid sequences set forth in SEQ ID NOS: 2, 10, and 12 or one or more variants having at least 80% amino acid sequence identity thereto; (3) the amino acid sequences set forth in SEQ ID NOS: 3, 6, and 11 or one or more variants having at least 80% amino acid sequence identity thereto; or (4) the amino acid sequences set forth in SEQ ID NOS: 4, 19, and 20 or one or more variants having at least 80% amino acid sequence identity thereto. Accordingly it will be appreciated that in certain embodiments provided herein the first peptide composition need not comprise all 21 of the herein disclosed SARS-CoV-2 Spike protein S1 or S2 region CD8+ T-cell epitope peptides of SEQ ID NOS: 1-20 and 39, or of SEQ ID NOS: 1-21.

In certain embodiments of the herein described methods, the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope may, for example, comprise at least: (1) the amino acid sequences set forth in SEQ ID NOS: 1, 3, and 5 or one or more variants having at least 80% amino acid sequence identity thereto; (2) the amino acid sequences set forth in SEQ ID NOS: 2, 4, and 7 or one or more variants having at least 80% amino acid sequence identity thereto; (3) the amino acid sequences set forth in SEQ ID NOS: 8, 10, and 12 or one or more variants having at least 80% amino acid sequence identity thereto; (4) the amino acid sequences set forth in SEQ ID NOS: 9, 14, and 15 or one or more variants having at least 80% amino acid sequence identity thereto; (5) the amino acid sequences set forth in SEQ ID NOS: 6, 11, and 18 or one or more variants having at least 80% amino acid sequence identity thereto; (6) the amino acid sequences set forth in SEQ ID NOS: 13, 16, and 39 or one or more variants having at least 80% amino acid sequence identity thereto; or (7) the amino acid sequences set forth in SEQ ID NOS: 17, 19, and 20 or one or more variants having at least 80% amino acid sequence identity thereto. Accordingly it will be appreciated that in certain embodiments provided herein the first peptide composition need not comprise all 21 of the herein disclosed SARS-CoV-2 Spike protein S1 or S2 region CD8+ T-cell epitope peptides of SEQ ID NOS: 1-20 and 39, or of SEQ ID NOS: 1-21.

For use in the methods described herein, a biological sample may be obtained from a subject for determining the presence and level of a T-cell immune response indicator as presently disclosed. A suitable biological sample may comprise, for instance, a whole blood sample, or a bronchial fluid or pulmonary lavage sample, or a mucous or sputum sample, or other respiratory, hematopoietic, ocular, or digestive system sample that would be recognized as comprising lymphocytes including T lymphocytes, any of which may be obtained from a subject using standard methodologies (e.g., from a human or animal subject and, in preferred embodiments, a human) having or suspected of being at risk for having COVID-19 disease or for having been previously exposed to SARS-CoV-2, such as a patient who tests positive for SARS-CoV-2 or who presents clinically with COVID-19 or who otherwise may have one or more COVID-19 disease risk factors, as will be recognized by those skilled in the art (see, e.g., Jeon et al., J Med Internet Res 2020; 22(10):e20509). In certain embodiments the biological sample may comprise at least one of whole blood (optionally with an anticoagulant), or a cellular fraction of whole blood, or isolated peripheral blood white cells, or isolated peripheral blood mononuclear cells. Biological samples may be obtained from a subject at a first timepoint that is prior to administration to the subject of a COVID-19 treatment or vaccine, which biological sample may be useful diagnostically and/or as a control for establishing baseline (i.e., pre-therapeutic) data; additionally or alternatively biological samples may be obtained from the subject at one or a plurality of second timepoints that are after administration to the subject of the COVID-19 treatment or vaccine. In certain embodiments a biological sample may be obtained from a subject before and/or after a SARS-CoV-2 vaccine has been administered to the subject, including a subject to whom the SARS-CoV-2 vaccine has been administered at at least one, two, three, four or more timepoints, typically separated in time from one another according to vaccination protocols. Non-limiting examples of SARS-CoV-2 vaccines include the Moderna mRNA-1273 vaccine (Moderna, Inc., Cambridge, MA), Pfizer-BioNTech COVID-19 vaccine (Pfizer Inc., NY), and ChAdOx1 nCoV-19 (AZD1222) vaccine (Folegatti et al., 2020 Lancet 396(10249):467-478).

### Detection of T-Cell Immune Response Indicators

In vitro antigen-specific T-cell responses are typically determined by comparisons of observed T-cell responses according to any of a number of described measurable T-cell functional parameters (e.g., proliferation, cytokine expression, cytokine biosynthesis, cytokine release, altered cell surface marker phenotype, etc.) that may be made between T-cells that are exposed to a cognate antigen in an appropriate context (e.g., the antigen used to prime or activate the T-cells, when presented by immunocompatible antigen-presenting cells) and T-cells from the same source population that are exposed instead to a structurally distinct or irrelevant control antigen. A response to the cognate antigen that is greater, with statistical significance, than the response to the control antigen signifies antigen-specificity.

The level of a secondary in vitro SARS-CoV-2-specific immune response may be determined by any one of numerous immunological methods described herein and/or routinely practiced in the art. The level of the secondary in vitro SARS-CoV-2-specific immune response may be determined at one or a plurality of timepoints, including timepoints that are prior to and/or following administration to the subject from whom the biological sample comprising T-cells and antigen-presenting cells are obtained (e.g., a subject known to have or suspected of having COVID-19 disease) of any one therapeutic and/or palliative treatments for COVID-19 disease and/or of a SARS-CoV-2 vaccine.

As described in the Examples, the presently disclosed composition comprising SARS-CoV-2 Spike protein S1 or S2 region CD8+ T-cell epitope-containing peptides (e.g., one or more peptides of SED ID NOS:1-20 and 39, or variants thereof as disclosed herein) was shown to be capable of stimulating detectable generation of a T-cell immune response indicator in a test incubation in vitro by a biological sample (whole blood) comprising T-cells and antigen-presenting cells from a subject previously exposed to SARS-CoV-2 but not in a control incubation using a sample from a subject who had not been exposed to the virus. The level of the T-cell immune response indicator that was detected was increased relative to a control level of the indicator that was obtained by incubating the biological sample in a control incubation that was otherwise identical to the test incubation except the SARS-CoV-2 peptide composition was omitted (“Nil”).

An increased level of a T-cell immune response indicator as provided herein thus may, in certain embodiments, take the form of a statistically significant increase in the level of the indicator that is detectable following incubation of T-cells and antigen presenting cells from a COVID-19-positive sample with the herein described SARS-CoV-2 Spike protein CD8+ T-cell epitope-containing peptide composition (SEQ ID NOS: 1-20 and 39) under conditions and for a time sufficient for specific antigen recognition by the T-cells, relative to the level of the indicator that is detectable under appropriate control conditions (e.g., without the SARS-CoV-2 Spike protein CD8+ T-cell peptide composition present, or using a COVID-19-negative sample).

By way of illustration and not limitation, in preferred embodiments, the T-cell immune response indicator that is generated by stimulation of T-cells with the SARS-CoV-2 Spike protein CD8+ T-cell epitope-containing peptide composition is at least one T-cell cytokine that is induced, expressed and/or released by the T-cells following incubation in vitro. Preferably the T-cell cytokine is selected from IL-1α, IL-1β, IL-2, IL-10, IL-12, IL-17, TNF-α, TNF-β, and IFN-γ. In certain preferred embodiments, released IFN-γ is the T-cell immune response indicator that is generated by stimulation of T-cells with the SARS-CoV-2 Spike protein CD8+ T-cell epitope-containing peptide composition. The contemplated embodiments are not, however, intended to be so limited, and therefore may include any of a wide variety of methodologies for assessing a biological sample as provided herein for its ability to mount a secondary in vitro antigen-specific response to the herein described SARS-CoV-2 Spike protein CD8+ T-cell epitope-containing peptide composition. Various assay configurations and techniques are known in the art (e.g., Current Protocols in Immunology, John Wiley & Sons, New York, N.Y.(2009)) and may be adapted to the present methods based on the instant disclosure. Levels of cytokines thus may be determined according to methods described herein and practiced in the art, including for example, ELISA, ELISPOT, intracellular cytokine staining, and/or flow cytometry.

In a preferred embodiment the T-cell immune response indicator is IFN-γ released by T-cells of the biological sample during the step of incubating the sample with the herein described SARS-CoV-2 Spike protein CD8+ T-cell epitope-containing peptide composition, and the level of released IFN-γ is determined immunochemically using any of a number of in vitro techniques by which IFN-γ is detected by determining detectable specific binding of a binding agent to the T-cell cytokine (i.e., IFN-γ). The binding agent may thus comprise at least one antibody that binds specifically to the cytokine (e.g., IFN-γ), which antibody may comprise at least one monoclonal antibody or which may instead comprise a polyclonal antibody. According to certain embodiments the T-cell immune response indicator is a cytokine that is released by T-cells and is detected by binding to an antibody that is immobilized on a solid phase.

An exemplary immunometric assay for IFN-γ is the QUANTIFERON® assay (available from QIAGEN, Germantown, MD), which is established as a gold standard for IFN-γ determination in a widely used test for tuberculosis (e.g., Pai et al., 2004 Lancet Infect Dis 4:761) and from which the quantitative detection of IFN-γ can be adapted for use with the herein described SARS-CoV-2 Spike protein CD8+ T-cell antigens instead of with tuberculosis antigens. (See also, e.g., Yun et al., 2014 J. Clin. Microbiol. 52:90; Belknap et al., 2014 Clin. Lab. Med. 34:337; Ferguson et al., 2015 Transplantation 99:1084; Ruan et al. 2014 Clin. Rheumatol. Epub PMID 25376466.)

A binding partner or an antibody is said to be “immunospecific,” “specific for” or to “specifically bind” an antigen of interest (e.g., a cytokine that is being assayed as a detectable T-cell immune response indicator, for instance, IFN-γ) if the antibody reacts at a detectable level with the antigen, preferably with an affinity constant, Kₐ, of greater than or equal to about 10⁴ M⁻¹, or greater than or equal to about 10⁵ M⁻¹, greater than or equal to about 10 6 M⁻¹, greater than or equal to about 10 7 M⁻¹, or greater than or equal to 10 8 M⁻¹. Affinity of an antibody for its cognate antigen is also commonly expressed as a dissociation constant K_{D}, and an antibody specifically binds to the antigen of interest if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N. Y. Acad. Sci. USA 51:660 (1949)) and by surface plasmon resonance (SPR; BIAcore™, Biosensor, Piscataway, NJ). For surface plasmon resonance, target molecules are immobilized on a solid phase and exposed to a binding partner (or ligand) in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

As used herein, the term “polyclonal antibody” refers to an antibody obtained from a population of antigen-specific antibodies that recognize more than one epitope of the specific antigen. “Antigen” or “immunogen” refers to a peptide, lipid, polysaccharide or polynucleotide which is recognized by the adaptive immune system. Antigens may be self or non-self molecules. Examples of antigens include, but are not limited to, bacterial cell wall components, pollen, and rh factor. The region of an antigen that is specifically recognized by a specific antibody, or by a specific T-cell receptor, is an “epitope” or “antigenic determinant.” A single antigen may have multiple epitopes.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The modifier “monoclonal” is not to be construed as requiring production of the antibody by any particular method. For example, monoclonal antibodies may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

### Nucleic Acids and Polynucleotides

SARS-CoV-2 Spike protein CD8+ T-cell epitope-containing polypeptides and peptides as provided herein, and encoding nucleic acid molecules and vectors, may be isolated and/or purified, e.g., from their natural environment, in substantially pure or homogeneous form, or, in the case of nucleic acid, free or substantially free of nucleic acid or genes of origin other than the sequence encoding a polypeptide with the desired function. Nucleic acid may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

The present invention thus further provides in certain embodiments an isolated nucleic acid encoding any of the SARS-CoV-2 Spike protein CD8+ T-cell epitope-containing peptides having the amino acid sequences set forth in SEQ ID NOS:1-21.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, Olignonucleotdies and Analogues: A Practical Approach, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present.

As will be understood by those skilled in the art, polynucleotides may include genomic sequences, extra-genomic and plasm id-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

Therefore, according to these and related embodiments, the present disclosure also provides polynucleotides encoding the SARS-CoV-2 Spike protein CD8+ T-cell epitope-containing peptides described herein. In certain embodiments, polynucleotides are provided that comprise some or all of a polynucleotide sequence encoding a peptide as described herein and complements of such polynucleotides.

In other related embodiments, polynucleotide variants may have substantial identity to a polynucleotide sequence encoding a SARS-CoV-2 Spike protein CD8+ T-cell epitope-containing peptide described herein. For example, a polynucleotide may be a polynucleotide comprising at least 80% sequence identity, preferably at least 85%, 90%, 95%, 96%, 97%, 98%, or 99% or higher, sequence identity compared to a reference polynucleotide sequence such as a sequence encoding a SARS-CoV-2 Spike protein CD8+ T-cell epitope-containing peptide described herein (e.g., a peptide having one of SEQ ID NOS:1-20 and 39 as its amino acid sequence), using the methods described herein, (e.g., BLAST analysis using standard parameters, as described below). One skilled in this art will recognize that these values can be appropriately adjusted to determine corresponding identity of polypeptides or peptides encoded by two nucleotide sequences by taking into account codon degeneracy, amino acid similarity, reading frame positioning and the like.

In another embodiment, polynucleotides are provided that are capable of hybridizing under moderate to high stringency conditions to a polynucleotide sequence encoding a SARS-CoV-2 Spike protein CD8+ T-cell epitope-containing peptide, or variant thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

As also described elsewhere herein, determination of the three-dimensional structures of representative SARS-CoV-2 Spike protein CD8+ T-cell epitope-containing peptides (e.g., SEQ ID NOS: 1-21 and 39 or variants thereof as provided herein, preferably SEQ ID NOS: 1-20 and 39 or variants thereof as provided herein) may be made through routine methodologies such that substitution, addition, deletion or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate polynucleotides encoding the amino acid sequence) within a peptide such that, for example, SARS-CoV-2 Spike protein CD8+ T-cell recognition is maintained.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in an intended recombinant DNA protocol to produce the presently disclosed SARS-CoV-2 Spike protein CD8+ T-cell epitope-containing peptides. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, C A (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a SARS-CoV-2 Spike protein CD8+ T-cell epitope-containing peptide as described herein. Some of these polynucleotides bear minimal sequence identity to the nucleotide sequence of the native or original polynucleotide sequence that encodes SARS-CoV-2 Spike protein CD8+ T-cell epitope-containing peptides described herein. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the SARS-CoV-2 Spike protein CD8+ T-cell epitope-containing peptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

According to certain related embodiments there is provided a recombinant host cell which comprises one or more constructs as described herein; a nucleic acid encoding a SARS-CoV-2 Spike protein CD8+ T-cell epitope-containing peptide or variant thereof; and a method of producing of the encoded product, which method comprises expression from encoding nucleic acid therefor. Expression may conveniently be achieved by culturing under appropriate conditions recombinant host cells containing the nucleic acid. Following production by expression, a SARS-CoV-2 Spike protein CD8+ T-cell epitope-containing peptide may be isolated and/or purified using any suitable technique, and then used as desired.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The recombinant expression of peptides in prokaryotic cells such as E. coli is well established in the art, as also is expression in eukaryotic cells in culture.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g., phage, or phagemid, as appropriate. For further details see, for example, Molecular Cloning: a Laboratory Manual: 4th edition, Green and Sambrook, 2012, Cold Spring Harbor Laboratory Press, Cold Spring Harbor, NY. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, N Y, 2015, or subsequent updates thereto.

The term “host cell” is used to refer to a cell into which has been introduced, or which is capable of having introduced into it, a nucleic acid sequence encoding one or more of the herein described SARS-CoV-2 Spike protein CD8+ T-cell epitope-containing peptides, and which further expresses or is capable of expressing a selected gene of interest, such as a gene encoding any herein described SARS-CoV-2 Spike protein CD8+ T-cell epitope-containing peptide. The term includes the progeny of the parent cell, whether or not the progeny are identical in morphology or in genetic make-up to the original parent, so long as the selected gene is present. Accordingly there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene. In one embodiment, the nucleic acid is integrated into the genome (e.g., chromosome) of the host cell. Integration may be promoted by inclusion of sequences which promote recombination with the genome, in accordance-with standard techniques.

The present disclosure also provides, in certain embodiments, a method which comprises using a construct as stated above in an expression system in order to express a particular polypeptide such as a SARS-CoV-2 Spike protein CD8+ T-cell epitope-containing peptide as described herein. The term “transduction” is used to refer to the transfer of genes from one bacterium to another, usually by a phage. “Transduction” also refers to the acquisition and transfer of eukaryotic cellular sequences by retroviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, e.g., Green and Sambrook, Molecular Cloning: a Laboratory Manual: 4th edition, 2012, Cold Spring Harbor Laboratory Press, Cold Spring Harbor, NY; Current Protocols in Molecular Biology, Ausubel et al. eds., John Wiley & Sons, N Y, 2015, or subsequent updates thereto. Such techniques can be used to introduce one or more exogenous DNA moieties into suitable host cells.

The term “transformation” as used herein refers to a change in a cell's genetic characteristics, and a cell has been transformed when it has been modified to contain a new DNA. For example, a cell is transformed where it is genetically modified from its native state. Following transfection or transduction, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “naturally occurring” or “native” when used in connection with biological materials such as nucleic acid molecules, polypeptides, host cells, and the like, refers to materials which are found in nature and are not manipulated by a human. Similarly, “non-naturally occurring” or “non-native” as used herein refers to a material that is not found in nature or that has been structurally modified or synthesized by a human.

It will be appreciated that the practice of the several embodiments of the present disclosure will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Green and Sambrook, Molecular Cloning: a Laboratory Manual: 4th edition, 2012, Cold Spring Harbor Laboratory Press, Cold Spring Harbor, NY; Current Protocols in Molecular Biology, Ausubel et al. eds., John Wiley & Sons, NY, 2015; Current Protocols in Molecular Biology or Current Protocols in Immunology, John Wiley & Sons, New York, NY (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Each embodiment described in this specification is to be applied mutatis mutandis to every other embodiment unless expressly stated otherwise.

Standard techniques may be used for biochemical and immunochemical and immunological assays, recombinant DNA, oligonucleotide synthesis, microbial and mammalian cell and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references in microbiology, molecular biology, biochemistry, molecular genetics, cell biology, virology and immunology techniques that are cited and discussed throughout the present specification. See, e.g., Sambrook, et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

Unless the context requires otherwise, throughout the present specification and claims, the word “comprise” and variations thereof, such as, “comprises” and “comprising” are to be construed in an open, inclusive sense, that is, as “including, but not limited to”. By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and may or may not be present depending upon whether or not they affect the activity or action of the listed elements.

In this specification and the appended claims, the singular forms “a,” “an” and “the” include plural references unless the content clearly dictates otherwise. As used herein, in particular embodiments, the terms “about” or “approximately” when preceding a numerical value indicates the value plus or minus a range of 5%, 6%, 7%, 8% or 9%. In other embodiments, the terms “about” or “approximately” when preceding a numerical value indicates the value plus or minus a range of 10%, 11%, 12%, 13% or 14%. In yet other embodiments, the terms “about” or “approximately” when preceding a numerical value indicates the value plus or minus a range of 15%, 16%, 17%, 18%, 19% or 20%.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### EXAMPLES

### Example 1

### Identification of Class I MHC-Binding Oligopeptides in SARS-Cov-2 Spike Protein S1 and S2 Regions and Demonstration of Covid-19-Immune CD8+ T-Cell Stimulation by Secondary In Vitro T-Cell Response to an Artificial Peptide Composition

The reference amino acid sequence (SEQ ID NO: 37, 1273 amino acids) of the full-length precursor of SARS-CoV-2 Spike glycoprotein (www_dot_uniprot.org/uniprot/P0DTC2) is shown in FIG. 1. The amino acid sequence of the spike protein receptor binding domain (RBD), amino acids 319-541, is shown in FIG. 2.

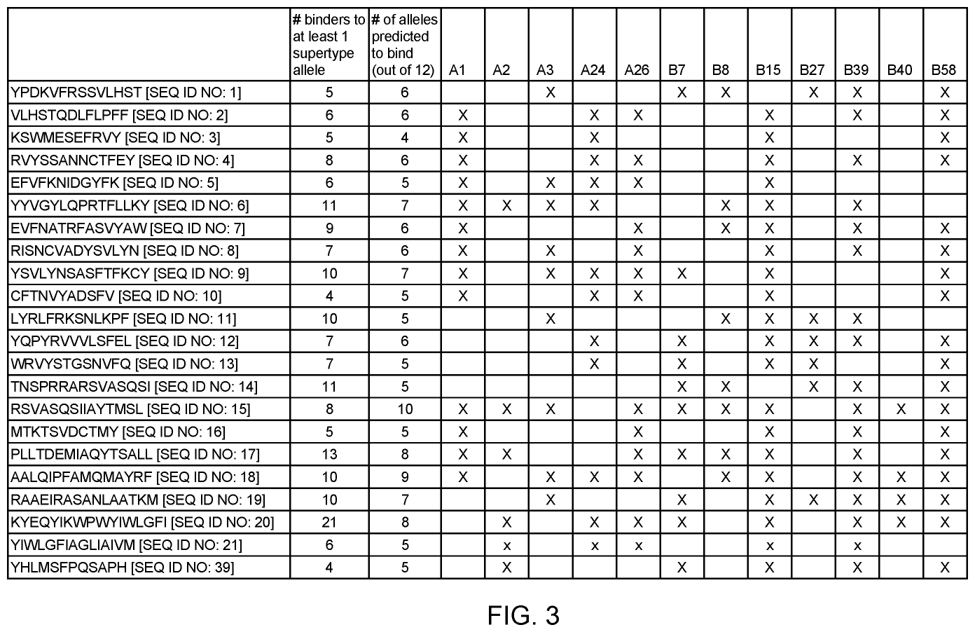

SARS-CoV-2 spike protein S1 and S2 region peptides recognized by CD8+ T-cells (SEQ ID NOS: 1-21 and 39) were identified based on in silico modeling of short peptide binding to MHC class I molecules (FIG. 3), which are known to provide the immunological context in which non-self antigens are presented to CD8+ T-cells, including CD8+ memory cells and cytotoxic lymphocytes (CTL). Class I binding of putative CD8+ T-cell epitope-containing oligopeptides of 11-17 amino acids in length was modeled using the binding prediction tool NetMHC 4.0. Candidate epitopes (SEQ ID NOS: 1-21 and 39) were identified in spike protein S1 and S2 region peptide sequences from two strains of SARS-CoV-2 (Wuhan and Bei strains) based on modeled binding to the indicated allelic forms of class I MHC (HLA) antigens (FIG. 3), and the selected peptides (Table 1) were found to exhibit 99% amino acid sequence conservation between these two strains while possessing less than 50% sequence homology with polypeptide sequences found in the common human seasonal coronaviruses (HCoV) 229E, NL63, OC43, and HKU1. The amino acid sequence position numbers in the full-length reference sequence (SEQ ID NO: 37) for each of SEQ ID NOS: 1-21 and 39 are presented above (see “Brief Description of the Sequences”), as also are the amino acid sequence position numbers for each of the overlapping CD4+ T-cell epitope-containing RBD peptides.

TABLE 1

| | # binders | | | | | | | | | | | | | |
|---|---|---|---|---|---|---|---|---|---|---|---|---|---|---|
| | to at | # of alleles | | | | | | | | | | | | |
| | least 1 | predicted to | | | | | | | | | | | | |
| | supertype | bind (out of | | | | | | | | | | | | |
| | allele | 12) | A1 | A2 | A3 | A24 | A26 | B7 | B8 | B15 | B27 | B39 | B40 | B58 |
| YPDKVFRSSVLHST | 5 | 6 | | | X | | | X | X | | X | X | | X |
| [SEQ ID NO: 1] | | | | | | | | | | | | | | |
| VLHSTQDLFLPFF | 6 | 6 | X | | | X | X | | | X | | X | | X |
| [SEQ ID NO: 2] | | | | | | | | | | | | | | |
| KSWMESEFRVY | 5 | 4 | X | | | X | | | | X | | | | X |
| [SEQ ID NO: 3] | | | | | | | | | | | | | | |
| RVYSSANNCTFEY | 8 | 6 | X | | | X | X | | | X | | X | | X |
| [SEQ ID NO: 4] | | | | | | | | | | | | | | |
| EFVFKNIDGYFK | 6 | 5 | X | | X | X | X | | | X | | | | |
| [SEQ ID NO: 5] | | | | | | | | | | | | | | |
| YYVGYLQPRTFLLKY | 11 | 7 | X | X | X | X | | | X | X | | X | | |
| [SEQ ID NO: 6] | | | | | | | | | | | | | | |
| EVFNATRFASVYAW | 9 | 6 | X | | | | X | | X | X | | X | | X |
| [SEQ ID NO: 7] | | | | | | | | | | | | | | |
| RISNCVADYSVLYN | 7 | 6 | X | | X | | X | | | X | | X | | X |
| [SEQ ID NO: 8] | | | | | | | | | | | | | | |
| YSVLYNSASFTFKCY | 10 | 7 | X | | X | X | X | X | | X | | | | X |
| [SEQ ID NO: 9] | | | | | | | | | | | | | | |
| CFTNVYADSFV | 4 | 5 | X | | | X | X | | | X | | | | X |
| [SEQ ID NO: 10] | | | | | | | | | | | | | | |
| LYRLFRKSNLKPF | 10 | 5 | | | X | | | | X | X | X | X | | |
| [SEQ ID NO: 11] | | | | | | | | | | | | | | |
| YQPYRVVVLSFEL | 7 | 6 | | | | X | | X | | X | X | X | | X |
| [SEQ ID NO: 12] | | | | | | | | | | | | | | |
| WRVYSTGSNVFQ | 7 | 5 | | | | X | | X | | X | X | | | X |
| [SEQ ID NO: 13] | | | | | | | | | | | | | | |
| TNSPRRARSVASQSI | 11 | 5 | | | | | | X | X | | X | X | | X |
| [SEQ ID NO: 14] | | | | | | | | | | | | | | |
| RSVASQSIIAYTMSL | 8 | 10 | X | X | X | | X | X | X | X | | X | X | X |
| [SEQ ID NO: 15] | | | | | | | | | | | | | | |
| MTKTSVDCTMY | 5 | 5 | X | | | | X | | | X | | X | | X |
| [SEQ ID NO: 16] | | | | | | | | | | | | | | |
| PLLTDEMIAQYTSALL | 13 | 8 | X | X | | | X | X | X | X | | X | | X |
| [SEQ ID NO: 17] | | | | | | | | | | | | | | |
| AALQIPFAMQMAYRF | 10 | 9 | X | | X | X | X | | X | X | | X | X | X |
| [SEQ ID NO: 18] | | | | | | | | | | | | | | |
| RAAEIRASANLAATKM | 10 | 7 | | | X | | | X | | X | X | X | X | X |
| [SEQ ID NO: 19] | | | | | | | | | | | | | | |
| KYEQYIKWPWYIWLGFI | 21 | 8 | | X | | X | X | X | | X | | X | X | X |
| [SEQ ID NO: 20] | | | | | | | | | | | | | | |
| YIWLGFIAGLIAIVM | 6 | 5 | | X | | X | X | | | X | | X | | |
| [SEQ ID NO: 21] | | | | | | | | | | | | | | |
| YHLMSFPQSAPH | 4 | 5 | | X | | | | X | | X | | X | | X |
| [SEQ ID NO: 39] | | | | | | | | | | | | | | |

Synthetic peptides were generated from the herein disclosed amino acid sequences (SEQ ID NOS: 1-36 and 39). Separate one (1) mL whole blood samples were drawn from previously confirmed SARS-CoV-2 positive and negative donors, and each sample was admixed with one of three Spike protein-derived peptide sets. To a first incubation test mixture for each blood sample was added the set of CD8 peptides (SEQ ID NOS: 1-20 and 39) alone, to a second incubation test mixture for each blood sample was added the set of CD4 peptides (SEQ ID NOS: 22-36) alone, and to a third incubation test mixture for each blood sample was added the combination of the CD4 and CD8 peptide sets (SEQ ID NOS: 1-20, 22-36, and 39). The CD4 peptide set (SEQ ID NOS: 22-36) consisted of 15 overlapping peptides from the Receptor Binding domain (RBD) of the spike protein (see “Brief Description of the Sequences”) The presence of CD4 T-cell epitopes in the RBD has been reported (Iyer et al. 2020 Science Immunology 8 Oct. 2020:Vol. 5, Issue 52, eabe0367; Su et al. 2020 Vaccine 2020 Jul. 6; 38(32): 5071-5075).

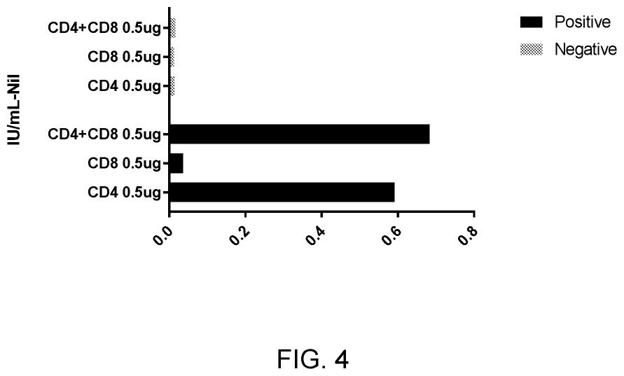

Following stimulation of the admixed incubation mixtures containing the blood samples plus the indicated peptide sets by incubation at 37±1° C., the resulting plasma was separated and harvested and T-cell mediated responses to SARS-CoV-2 were assessed through the measurement of the resulting cytokine (IFN-γ) that was released upon stimulation with these peptides. The IFN-γ that was produced was measured using QuantiFERON® (QIAGEN, Germantown, MD) ELISA according to the manufacturer's recommendations. The results for the IU/mL of the IFN-γ after Nil (background) subtraction are shown in FIG. 4 and summarized in Table 2.

TABLE 2
Mean IU/mL-Nil values for each peptide set.

| | Positive | Negative |
|---|---|---|
| CD4 0.5 ug | 0.591 | 0.015 |
| CD8 0.5 ug | 0.036 | 0.013 |
| CD4 + CD8 0.5 ug | 0.683 | 0.018 |

The CD8+ T-cell epitope-containing spike protein peptides (SEQ ID NOS: 1-20 and 39) elicited a T-cell IFNγ response specifically in the samples obtained from SARS-CoV-2 positive donors, as shown in FIG. 4 and Table 2. In addition, the CD8+ T-cell epitope-containing peptides (SEQ ID NOS: 1-20 and 39) enhanced the T-cell IFNγ response to CD4+ T-cell epitope-containing peptides (SEQ ID NOS: 22-36).

### Example 2

### Measuring T Cell-Mediated Response in SARS-Cov-2 Vaccinated Donors with Spike RBD CD4 Peptides and CD8 Peptides

This example describes measurement of secondary in vitro T cell-mediated immune responses by T cells present in biological samples obtained from SARS-CoV-2 vaccinated subjects. Immunoresponsiveness was detected using the herein disclosed sets of peptides containing SARS-CoV-2 epitopes for CD4+ T cells, which peptides were derived from the Receptor Binding Domain (RBD) of the Spike protein (SEQ ID NOS: 22-36) as described herein, and SARS-CoV-2 epitopes for CD8+ T cells, which peptides were derived from the Spike protein of SARS-CoV-2 (SEQ ID NOS: 1-20 and 39) as also described herein.

The CD4 epitope-bearing peptides alone (“Ag1”, SEQ ID NOS: 22-36), or a combination of CD4 and CD8 epitope-bearing epitopes (“Ag2”, SEQ ID NOS: 1-20, 22-36, and 39) were spray-dried onto the walls of QuantiFERON® blood collection tubes designated SARS-CoV-2 Ag1 and SARS-CoV-2 Ag2, respectively.

SARS-CoV-2 vaccine (Moderna mRNA-1273, Moderna, Cambridge, MA) was administered to human study subjects according to the manufacturer's recommendations, and blood samples were collected from seven donor subjects on days 4 and 11 post-vaccination, and from two other donors on day 7 post-vaccination. Blood was collected in a 9 mL generic lithium heparin tube following an approved IRB protocol (Qiagen Sciences, Inc., Germantown, MD).

The blood draw timepoints of the 10 donors assessed in this study are shown in Table 3.

TABLE 3
Blood Draw Timepoints for donors

| Donors | Blood Draw/Testing Date |
|---|---|
| Donors 1-7 | Days 4 and 11 after first dose of vaccine |
| Donor 8 | Day 7 after first dose of vaccine |
| Donor 9 | Days 0 and 7 after first dose of vaccine |
| Donor 10 | Day 22 after first dose of vaccine and |
| | Day 7 after Second dose of Vaccine |

One milliliter aliquots of each whole blood sample were added to the QuantiFERON® SARS-CoV-2 Ag1 (SEQ ID NOS: 22-36) and SARS-CoV-2 Ag2 (SEQ ID NOS: 1-20, 22-36, and 39) tubes, and to control tubes. QuantiFERON® Nil tubes (lacking any stimulatory antigen) were used as negative controls and QuantiFERON® Mitogen tubes (containing the non-specific T cell mitogen phytohemagglutinin (PHA)) were used as positive controls for T cell stimulation. After blood samples were added, the tubes were shaken 10 times to allow the blood to sufficiently dissolve the antigens (or mitogen) from the tube walls, and were then incubated at 37° C. for 16-24 hours to permit stimulation of T cells. Following stimulation at 37° C., the incubation tubes were centrifuged at 2500×g for 15 minutes. Plasma (supernatant fluid) was harvested from each tube and aliquots were assessed for IFN-γ release using the QuantiFERON® enzyme-linked immunosorbent assay (ELISA, QIAGEN) according to the manufacturer's instructions.

Separately, aliquots of plasma samples recovered from the whole blood samples remaining in the generic lithium heparin blood collection tubes were tested for the presence of SARS-CoV-2-reactive antibodies using the QIAreach® Anti-SARS-CoV-2 Total test (QIAGEN) according to the manufacturer's instructions.

### Results:

The IFN-γ-specific ELISA results were analyzed using QuantiFERON® R&D software to generate international units per milliliter (IU/mL) values for detectable interferon-gamma (IFNγ). The IFN-γ IU/mL values detected in samples incubated in QuantilFERON® (QFN) Nil tubes were subtracted from the IFN-γ IU/mL values obtained for samples that had been incubated in QuantiFERON® SARS-CoV-2 Ag1, QFN SARs-CoV-2Ag2, and QFN Mitogen tubes, to correct for detectable background signal or nonspecifically elicited IFN-γ in the blood samples.

The Nil-subtracted IFN-γIU/mL values (T cell responses) and the QIAreach® CoV-2 Total (antibody responses) results for samples collected from Donors 1-7 are shown in Table 4, the results for Donors 8 and 9 are shown in Table 5, and the results for Donor 10 are shown in Table 6.

TABLE 4
QuantiFERON ® SARS-CoV-2 and QIAreach ® CoV-2 Total
Results for Donors 1-7

| | Day 4 | | | | Day 11 | | | |
|---|---|---|---|---|---|---|---|---|
| | QuantiFERON ® | | | | QuantiFERON ® | | | |
| | SARS-CoV-2 | | | | SARS-CoV-2 | | | |
| Donor | Ag1- | Ag2- | Mit- | QIAreach ® | Ag1- | Ag2- | Mit- | QIAreach ® |
| ID | Nil | Nil | Nil | CoV2T | Nil | Nil | Nil | CoV2T |
| Donor 1 | −0.01 | −0.01 | 7.19 | Negative | 4.76 | 8.40 | >10 | Positive |
| Donor 2 | 0.00 | 0.00 | 5.74 | Negative | 4.50 | 4.41 | >10 | Positive |
| Donor 3 | −0.09 | 0.03 | 7.07 | Negative | 1.12 | 3.44 | >10 | Positive |
| Donor 4 | 0.02 | 0.05 | 5.78 | Negative | 3.35 | 7.73 | >10 | Positive |
| Donor 5 | 0.00 | 0.00 | 7.13 | Negative | 0.36 | 0.53 | >10 | Positive |
| Donor 6 | 0.01 | 0.01 | 6.59 | Negative | 0.54 | 0.48 | >10 | Positive |
| Donor 7 | −3.44* | −3.44* | 3.01 | Negative | 1.46 | 6.20 | >10 | Positive |

*High negative value for the Donor 7 sample collected at Day 4 post-vaccination is attributed to high IFN-γ levels in the negative control Nil (background) tube, likely caused by sample contamination; the IFNγ level recorded for the Nil tubes incubated with the Donor 7 sample that was collected subsequently on Day 11 did not yield comparably high background levels.

TABLE 5
QuantiFERON ® SARS-CoV-2 and QIAreach ® CoV-2 Total
Results for Donors 8-9

| | Day 0 | | | | Day 7 | | | |
|---|---|---|---|---|---|---|---|---|
| | QuantiFERON | | | | QuantiFERON | | | |
| | SARS-CoV-2 | | | | SARS-CoV-2 | | | |
| Donor | Ag1- | Ag2- | Mit- | QIAReach | Ag1- | Ag2- | Mit- | QIAReach |
| ID | Nil | Nil | Nil | CoV2T | Nil | Nil | Nil | CoV2T |
| Donor 8 | n/a | n/a | n/a | n/a | 0.61 | 0.33 | 7.13 | Negative |
| Donor 9 | −0.01 | −0.01 | 6.19 | Negative | 0.27 | 0.39 | >10 | Negative |

TABLE 6
QuantiFERON ® SARS-CoV-2 and QIAreach ® CoV2T
Results for Donors 10

| | Day 22 (First-dose) | | | | Day 7 (Second Dose) | | | |
|---|---|---|---|---|---|---|---|---|
| | QuantiFERON | | | QIAReach | QuantiFERON | | | QIAReach |
| | SARS-CoV-2 | | | SARS- | SARS-CoV-2 | | | SARS- |
| Donor | Ag1- | Ag2- | Mit- | CoV-2 | Ag1- | Ag2- | Mit- | CoV-2 |
| ID | Nil | Nil | Nil | Total | Nil | Nil | Nil | Total |
| Donor | 0.17 | 0.24 | >10 | Positive | 0.48 | 0.86 | >10 | Positive |
| 10 | | | | | | | | |

The samples from donors (1-7) that were collected for immunological testing on Days 4 and 11 post-vaccination showed no T-cell or antibody response after 4 days, but exhibited a T-cell mediated response measured by IFN-gamma release as well as an anti-SARS-CoV-2 antibody response 11 days after vaccination (Table 4). The samples from two donors (8-9) that were collected for immunological testing 7 days post-vaccination showed T-cell mediated responses as measured by IFN-γ release but no detectable anti-SARS-CoV-2 antibody response (Table 5). In most of the samples that were collected and immunologically tested, there was a notable increase in the level of the detectable IFN-γ (T-cell) response to stimulation with both CD4 and CD8 T cell-reactive peptides (Ag2 tubes), relative to the level in response to stimulation with only CD4 T cell-reactive peptides (Ag1 tubes), indicating a significant contribution of CD8 T cells to the detectable IFNγ responses.

Additionally, a blood sample from a tenth donor was collected and tested on day 22 after the first dose of vaccination, and another blood sample was collected from this donor and tested on day 7 following the administration of a second vaccine dose (Table 6). The results showed elevated secondary in vitro T-cell immune response (IFN-γ) levels (especially the CD8 response in the Ag2 group) by the samples collected after the subject had received the second dose of the vaccine, relative to those observed for the samples collected after the first vaccine dose. The blood samples collected from this donor on both test dates returned positive anti-SARS-CoV-2 antibody results. (Table 6).

The results show that the presently disclosed compositions for diagnosis or prognosis of coronavirus disease 2019 (Covid-19), or for detecting an antigen-specific T cell-mediated immune response to severe acute respiratory syndrome coronavirus 2 (SARS-CoV-2), stimulated antigen-specific secondary in vitro CD4 and CD8 T cell-mediated immune responses by immune cells present in biological samples obtained from subjects to whom a SARS-CoV-2 vaccine had been administered. The SARS-CoV-2 Ag1 and Ag2 tubes elicited a selective T-cell mediated response in samples collected from 2/2 donors tested on day 7 post-vaccination and from 7/7 donors tested on day 11 post-vaccination. There was no T-cell mediated response seen in samples collected from any of these seven donors at day 4 post-vaccination, suggesting that the development of a detectable T-cell mediated response to SARS-CoV-2 may occur between 5 and 7 days post-vaccination.

The Ag2 tubes, which contained (i) peptides (SEQ ID NOS: 22-36) bearing the CD4 epitopes from the SARS-CoV-2 Spike protein RBD region (which peptides were also present by themselves in Ag1 tubes), along with (ii) peptides (SEQ ID NOS: 1-20 and 39) bearing the CD8 epitopes from the SARS-CoV-2 Spike protein, stimulated augmented in vitro IFNγ responses by immune cells present in biological samples collected from 7 of the 10 donors that were tested (donors 1, 3, 4, 5, 7, 9 and 10) (Tables 4-6). Furthermore, the Ag2/CD8 responses were augmented in the samples collected from donor 10 following the second vaccination dose (Table 7).

TABLE 7
Difference in IU/mL Values between Ag1 and Ag2 tubes

| | | (Ag2-Ag1) |
|---|---|---|
| Time Post Vaccine | Donor ID | IU/mL |
| Dose 1, Day 11 | Donor 1 | 3.64 |
| | Donor 3 | 2.32 |
| | Donor 4 | 4.38 |
| | Donor 5 | 0.17 |
| | Donor 7 | 4.74 |
| Dose 1, Day 7 | Donor 9 | 0.12 |
| Dose 1, Day 22 | Donor 10 | 0.07 |
| Dose 2, Day 7 | Donor 10 | 0.38 |

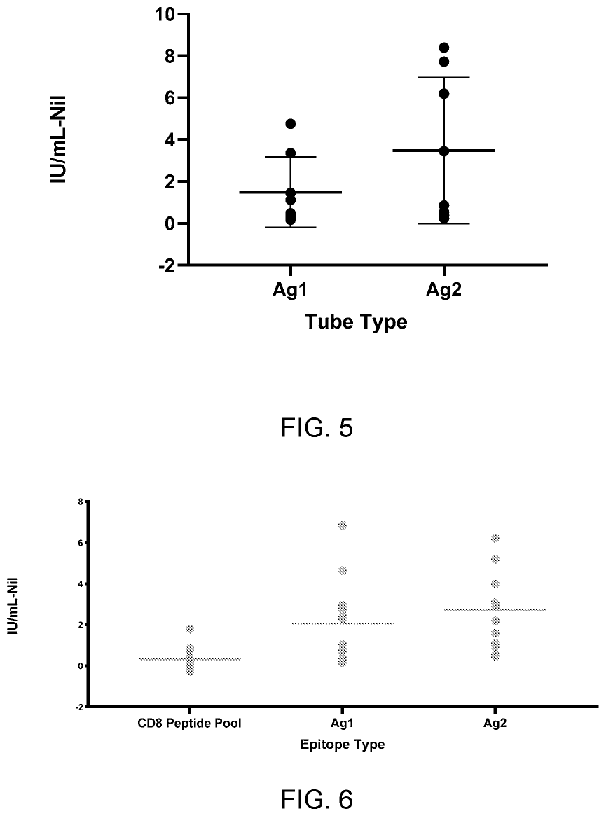

The mean secondary in vitro T cell-mediated IFNγ immune response levels (IU/mL) response levels that were recorded by testing samples from vaccinated donors 1, 3, 4, 5, 7, 9 and 10 for Ag1 and Ag2 are shown in FIG. 5.

### Example 3

### Measuring T Cell-Mediated Response in SARS-Cov-2 Vaccinated Donors with Spike RBD CD4 Peptides and Sub-Pools of CD8 Peptides

This example describes measurement of secondary in vitro T cell-mediated immune responses to subsets (also referred to as “sub-pools”) of three CD8+ T cell epitope-containing oligopeptides selected from within the larger set of herein disclosed SARS-CoV-2 Spike protein peptides containing epitopes for CD8+ T cells (SEQ ID NOS: 1-21 and 39). Methodology was essentially as described above in Example 2, except as otherwise noted in this Example. Subsets (referred to as “sub-pools”) of three CD8 peptides were prepared as shown in Table 8.

TABLE 8
SARS-COV-2 Spike Protein Sub-Pools

| Sub-Pool | CD8 peptide | CD8 peptide | CD8 peptide |
|---|---|---|---|
| 1 | p01 | p03 | p05 |
| | SEQ ID NO: 1 | SEQ ID NO: 3 | SEQ ID NO: 5 |
| 2 | p02 | p09 | p10 |
| | SEQ ID NO: 2 | SEQ ID NO: 10 | SEQ ID NO: 12 |
| 3 | p03 | p021 | p22 |
| | SEQ ID NO: 3 | SEQ ID NO: 6 | SEQ ID NO: 11 |
| 4 | p04 | p017 | p19 |
| | SEQ ID NO: 4 | SEQ ID NO: 19 | SEQ ID NO: 20 |

Secondary in vitro T cell immunoresponsiveness was detected following incubation of T cells with and without the herein disclosed “Ag1” Spike protein RBD peptides containing SARS-CoV-2 epitopes for CD4+ T cells (SEQ ID NOS: 22-36), along with none, some subsets (“sub-pools”), or 21 of the presently disclosed SARS-CoV-2 Spike protein epitopes for CD8+ T cells (SEQ ID NOS: 1-20 and 39).

The following combinations of SARS-CoV-2 CD8+ T cell epitope-bearing peptides (SEQ ID NOS: 1-20 and 39) and/or CD4+ T cell epitope-bearing peptides (SEQ ID NOS: 22-36) were spray-dried onto the walls of QuantiFERON® blood collection tubes:

TABLE 9
SARS-COV-2 Spike Protein Peptide Combinations

| Group | CD8+ epitope peptides | CD4+ epitope peptides |
|---|---|---|
| 1 (“Ag1”) | none | SEQ ID NOS: 22-36 |
| 2 (“Ag2”) | SEQ ID NOS: 1-20 and 39 | SEQ ID NOS: 22-36 |
| 3 | SEQ ID NOS: 1-20 and 39 | none |
| 4 | CD8 sub-pool 1 | none |
| | SEQ ID NOS: 1, 3, 5 | |
| 5 | CD8 sub-pool 2 | none |
| | SEQ ID NOS: 2, 10, 12 | |
| 6 | CD8 sub-pool 3 | none |
| | SEQ ID NOS: 3, 6, 11 | |
| 7 | CD8 sub-pool 4 | none |
| | SEQ ID NOS: 4, 19, 20 | |
| 8 | CD8 sub-pool 1 | SEQ ID NOS: 22-36 (Ag1) |
| | SEQ ID NOS: 1, 3, 5 | |
| 9 | CD8 sub-pool 2 | SEQ ID NOS: 22-36 (Ag1) |
| | SEQ ID NOS: 2, 10, 12 | |
| 10 | CD8 sub-pool 3 | SEQ ID NOS: 22-36 (Ag1) |
| | SEQ ID NOS: 3, 6, 11 | |
| 11 | CD8 sub-pool 4 | SEQ ID NOS: 22-36 (Ag1) |
| | SEQ ID NOS: 4, 19, 20 | |
| 12 | None (Nil, negative control) | None (Nil, negative control) |
| 13 | None + Mitogen | None + Mitogen |
| | (positive control) | (positive control) |

SARS-CoV-2 vaccine (Moderna mRNA-1273, Moderna, Cambridge, MA) was administered to 12 human study subjects according to the manufacturer's recommendations. Blood was collected in 9 mL generic lithium heparin tubes following an approved IRB protocol (Qiagen Sciences, Inc., Germantown, MD). Blood samples were collected from the 12 vaccinated donor subjects and from two additional convalescent (i.e., previously exposed to SARS-CoV-2) subjects (Donors 11 and 12) at the following timepoints.

TABLE 10
Blood Draw Timepoints for Donors

| Donors | Blood Draw/Testing Date |
|---|---|
| Donor 1 | Day 8 post second dose |
| Donors 2, 5, 6 | Day 7 post second dose |
| Donors 3, 7, 8, 9, | Day 11 post second dose |
| 10, 13, 14 | |
| Donors 11 and 12 | Convalescent |
| Donor 4 | Day 35 post second dose |

One milliliter aliquots of each whole blood sample were added to QuantiFERON® SARS-CoV-2 tubes prepared with Spike protein peptides as indicated in Table 9, Groups 1-11. QuantiFERON® Nil tubes lacking any stimulatory antigen (Group 12)) were used as negative controls and QuantiFERON® Mitogen tubes containing the non-specific T cell mitogen phytohemagglutinin (PHA) (Group 13) were used as positive controls for T cell stimulation. After blood samples were added, the tubes were shaken 10 times to allow the blood to sufficiently dissolve the antigens (or mitogen) from the tube walls to obtain incubation mixtures, which were then incubated at 37° C. for 16-24 hours to permit stimulation of T cells. Following stimulation at 37° C., the incubation tubes were centrifuged at 2500×g for 15 minutes. Plasma (supernatant fluid) was harvested from each tube and aliquots were assessed for IFN-γ release using the QuantiFERON® enzyme-linked immunosorbent assay (ELISA, QIAGEN) according to the manufacturer's instructions.

Results: IFN-γ release was detected in supernatant fluids using the QuantiFERON® ELISA. The results were analyzed using QuantiFERON® R&D software to generate IU/mL values. The IU/mL values for QuantiFERON® (QFN) Nil (Group 12) were subtracted from the IU/mL values for QuantiFERON® SARS Ag1 (Group 1), QuantiFERON® SARS Ag2 (Group 2) and QuantiFERON® Mitogen (PHA, Group 13) tubes to adjust for background or nonspecific IFN-γ in blood samples. The Nil subtracted IU/mL values that were obtained using samples from the 14 donors are shown in Table 11.

TABLE 11
QuantiFERON ® SARS-COV-2 Results for 14 Donors

| | Donor | Donor | Donor | Donor | Donor | Donor | Donor | Donor | Donor | Donor | Donor | Donor | Donor | Donor |
|---|---|---|---|---|---|---|---|---|---|---|---|---|---|---|
| | 1 | 2 | 3 | 4 | 5 | 6 | 7 | 8 | 9 | 10 | 11 | 12 | 13 | 14 |
| CD8 | 0.76 | 0.26 | 0.38 | 0.01 | 0.05 | 1.79 | 0.06 | −0.26 | 0.08 | 0.29 | 0.05 | 0.25 | 0.85 | 0.05 |
| Peptide | | | | | | | | | | | | | | |
| Pool | | | | | | | | | | | | | | |
| CD8 | 0.03 | 0.08 | −0.02 | 0 | 0 | 0 | −0.01 | −0.39 | −0.02 | 0 | −0.02 | 0 | 0.09 | −0.01 |
| Pep | | | | | | | | | | | | | | |
| Pool 1 | | | | | | | | | | | | | | |
| CD8 | 0.2 | −0.01 | 0.18 | 0 | 0 | 0 | −0.01 | −0.33 | −0.01 | 0.02 | −0.02 | 0.3 | −0.01 | −0.01 |
| Pep | | | | | | | | | | | | | | |
| Pool 2 | | | | | | | | | | | | | | |
| CD8 | 0.28 | 0 | 0.03 | 0 | 0.01 | 0.09 | 0.01 | −0.36 | −0.01 | 0 | −0.04 | −0.01 | −0.02 | −0.01 |
| Pep | | | | | | | | | | | | | | |
| Pool 3 | | | | | | | | | | | | | | |
| CD8 | 0.06 | 0.23 | 0.38 | 0 | 0 | 0.44 | 0 | −0.13 | 0.02 | 0.01 | −0.03 | −0.01 | 0.84 | 0 |
| Pep | | | | | | | | | | | | | | |
| Pool 4 | | | | | | | | | | | | | | |
| Ag1 | 4.64 | 2.74 | 2.28 | 0.26 | 2.39 | 2.96 | 0.73 | 1.05 | 2.92 | 6.84 | 0.73 | 0.16 | 0.77 | 0.38 |
| Ag1 + | 4.66 | 2.58 | 2.48 | 0.1 | 1.44 | 4.4 | 0.27 | 0.38 | 1.95 | 6.95 | 0.5 | 0.12 | 1.31 | 0.21 |
| CD8 | | | | | | | | | | | | | | |
| Pep | | | | | | | | | | | | | | |
| Pool 1 | | | | | | | | | | | | | | |
| Ag1 + | 4.83 | 2.21 | 2.99 | 0.12 | 1.94 | 3.54 | 0.34 | 0.22 | 2.01 | 8.06 | 0.47 | 0.4 | 0.93 | 0.24 |
| CD8 | | | | | | | | | | | | | | |
| Pep | | | | | | | | | | | | | | |
| Pool 2 | | | | | | | | | | | | | | |
| Ag1 + | 4.92 | 2.04 | 2.11 | 0.06 | 1.38 | 3.33 | 0.56 | 0.35 | 2.16 | 8.67 | 0.35 | 0.06 | 0.78 | 0.21 |
| CD8 | | | | | | | | | | | | | | |
| Pep | | | | | | | | | | | | | | |
| Pool 3 | | | | | | | | | | | | | | |
| Ag1 + | 5.16 | 2.37 | 2.9 | 0.12 | 1.54 | 4.01 | 0.77 | 0.34 | 2.06 | 9.09 | 0.54 | 0.07 | 1.86 | 0.2 |
| CD8 | | | | | | | | | | | | | | |
| Pep | | | | | | | | | | | | | | |
| Pool 4 | | | | | | | | | | | | | | |
| Ag2 | 6.22 | 3.98 | 3.09 | 0.52 | 2.87 | 5.2 | 0.94 | 1.6 | 3.05 | 6.21 | 0.94 | 1.09 | 2.18 | 0.46 |

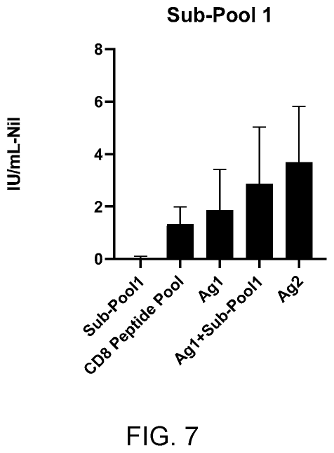

FIG. 6 shows the IFN-γ response (IU/mL-Nil) to CD8+ epitopes alone (SEQ ID NOS: 1-20 and 39; Group 3), CD4 epitopes alone (Ag1, SEQ ID NOS: 22-36) and CD4+-plus-CD8+ epitopes (Ag2, SEQ ID NOS: 1-20, 22-36, and 39) in QuantiFERON® tubes from all 14 donors. As shown in Table 11, Donor 13 showed a slight response to CD8 sub-pool 1 and a synergistic effect when responding to the CD8 sub-pool in combination with CD4 (“Ag1”) epitopes, while Donor 6 showed no response to CD8 sub-pool 1 alone but exhibited a synergistic response to CD8 sub-pool 1 in combination with CD4 (Ag1) epitopes, see Tables 11 and 12. The mean IFN-γ IU/mL responses for donors 6 and 13 are shown in FIG. 7.

TABLE 12
Mean IFN-γ (IU/mL-Nil) values for Donors 6 and 13

| | Donor 6 | Donor 13 |
|---|---|---|
| Sub-Pool 1 | 0 | 0.09 |
| CD8 Peptide Pool | 1.79 | 0.85 |
| Ag1 | 2.96 | 0.77 |
| Ag1 + Sub-Pool 1 | 4.4 | 1.31 |
| Ag2 | 5.2 | 2.18 |

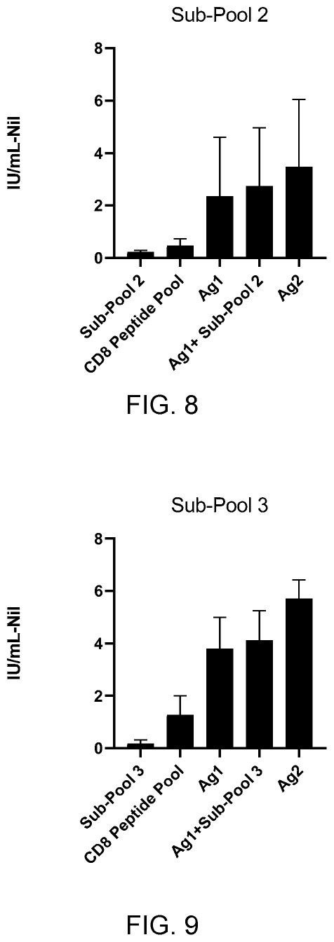

As shown in Table 13, T cells from Donors 1, 3, and 12 showed low IFN-γ responses to CD8 Sub-Pool 2 alone and showed additive effects in response to Sub-Pool 2 in combination with CD4 (Ag1) epitopes (SEQ ID NOS: 22-36). The mean IFN-γ IU/mL responses of T cells from donors 1, 3 and 12 to Sub-Pool 2 are shown in FIG. 8.

TABLE 13
Mean IFN-γ (IU/mL-Nil) values for Donors 1, 3,
and 12, showingr esponse to Sub-pool 2 alone
or in combination with CD4 epitopes in Ag1.

| | Donor 1 | Donor 3 | Donor 12 |
|---|---|---|---|
| Sub-Pool 2 | 0.2 | 0.18 | 0.3 |
| CD8 Peptide Pool | 0.76 | 0.38 | 0.25 |
| Ag1 | 4.64 | 2.28 | 0.16 |
| Ag1 + Sub-Pool 2 | 4.83 | 2.99 | 0.4 |
| Ag2 | 6.22 | 3.09 | 1.09 |

Donors 1 and Donor 6 showed responses to CD8 Sub-Pool 3 alone as shown in Table 14, and donor 6 showed a slight synergistic response to Sub-Pool 3 in combination with CD4 (Ag1) epitopes (SEQ ID NOS: 22-36). The mean IFN-γ IU/mL responses for donors 1 and 6 are shown in FIG. 9.

TABLE 14
Mean IFN-γ (IU/mL-Nil) values for Donors 1 and 6.

| | Donor 1 | Donor 6 |
|---|---|---|
| Sub-Pool 3 | 0.28 | 0.09 |
| CD8 Peptide Pool | 0.76 | 1.79 |
| Ag1 | 4.64 | 2.96 |
| Ag1 + Sub-Pool 3 | 4.92 | 3.33 |
| Ag2 | 6.22 | 5.2 |

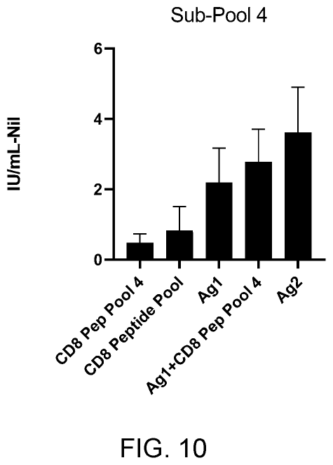

Donors 2, 3, 6 and 13 showed responses to CD8 Sub-Pool 4 alone and Donors 6 and 13 showed slight synergistic responses to sub-pool 4 in combination with CD4 (Ag1) epitopes (SEQ ID NOS: 22-36) as shown in Table 15. The mean IFN-γ (IU/mL-Nil) responses for donors 2, 3, 6 and 13 are shown in FIG. 10.

TABLE 15
Mean IFN-γ (IU/mL-Nil) values for Donors 2, 3, 6, and 12.

| | Donor | Donor | Donor | Donor |
|---|---|---|---|---|
| | 2 | 3 | 6 | 13 |
| CD8 Peptide Pool | 0.26 | 0.38 | 1.79 | 0.85 |
| CD8 Pep Pool 4 | 0.23 | 0.38 | 0.44 | 0.84 |
| Ag1 | 2.74 | 2.28 | 2.96 | 0.77 |
| Ag1 + CD8 Pep Pool 4 | 2.37 | 2.9 | 4.01 | 1.86 |
| Ag2 | 3.98 | 3.09 | 5.2 | 2.18 |

QuantiFERON® SARS-CoV-2 CD8+ T cell epitope-containing Spike protein peptides (SEQ ID NOS: 1-20 and 39) were shown to elicit secondary in vitro cell mediated immune responses by immune cells present in blood samples obtained from SARS-CoV-2 convalescent and vaccinated donors, when the CD8 epitopes were pooled as 21 epitopes (SEQ ID NOS: 1-20 and 39), or as sub-pools of three epitopes selected from among the 21 epitopes, either alone or in combination with CD4 epitopes. Not all 21 epitopes were needed to elicit a secondary in vitro T cell response, and certain epitopes more potently elicited responses in T cells from some donors as compared to others.

Secondary in vitro immune response testing was also conducted using the following additional CD8+ T cell epitope sub-pools, each containing three epitopes selected from among the 21 peptides disclosed herein as SEQ ID NOS: 1-20 and 39 (e.g., Table 1 and FIG. 3):

| Sub-pool | SEQ ID NOS: |
|---|---|
| 5 (same as sub-pool 1) | 1, 3, 5 |
| 6 | 2, 4, 7 |
| 7 | 8, 10, 12 |
| 8 | 9, 14, 15 |
| 9 | 6, 11, 18 |
| 10 | 13, 16, 39 |
| 11 | 17, 19, 20 |

The results indicated again that sub-pools of three CD8 epitopes were capable of eliciting a secondary in vitro T cell response and could enhance the level of response that was induced by CD4 (Ag1) epitopes. For a given sub-pool, the potency of the responses varied among T cells sourced from different donors.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/121,490, filed on Dec. 4, 2020, and U.S. Patent Application No. 63/150,890, filed on Feb. 18, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A composition for diagnosis or prognosis of coronavirus disease 2019 (Covid-19), or for detecting an antigen-specific T cell-mediated immune response to severe acute respiratory syndrome coronavirus 2 (SARS-CoV-2), comprising 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 and 39, preferably in SEQ ID NOS: 1-20 and 39:
SEQ ID NO: 1
YPDKVFRSSVLHST,
SEQ ID NO: 2
VLHSTQDLFLPFF,
SEQ ID NO: 3
KSWMESEFRVY,
SEQ ID NO: 4
RVYSSANNCTFEY,
SEQ ID NO: 5
EFVFKNIDGYFK,
SEQ ID NO: 6
YYVGYLQPRTFLLKY,
SEQ ID NO: 7
EVFNATRFASVYAW,
SEQ ID NO: 8
RISNCVADYSVLYN,
SEQ ID NO: 9
YSVLYNSASFTFKCY,
SEQ ID NO: 10
CFTNVYADSFV,
SEQ ID NO: 11
LYRLFRKSNLKPF,
SEQ ID NO: 12
YQPYRVVVLSFEL,
SEQ ID NO: 13
WRVYSTGSNVFQ,
SEQ ID NO: 14
TNSPRRARSVASQSI,
SEQ ID NO: 15
RSVASQSIIAYTMSL,
SEQ ID NO: 16
MTKTSVDCTMY,
SEQ ID NO: 17
PLLTDEMIAQYTSALL,
SEQ ID NO: 18
AALQIPFAMQMAYRF,
SEQ ID NO: 19
RAAEIRASANLAATKM,
SEQ ID NO: 20
KYEQYIKWPWYIWLGFI,
SEQ ID NO: 21
YIWLGFIAGLIAIVM,
and
SEQ ID NO: 39
YHLMSFPQSAPH,
or one or more variants thereof having at least 80% amino acid sequence identity to the amino acid sequences set forth in SEQ ID NOS:1-21 and 39.

2. The composition of claim 1 which further comprises 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36:
SEQ ID NO: 22
RVQPTESIVRFPNITNLCPFGEVEN,
SEQ ID NO: 23
NLCPFGEVFNATRFASVYAWNRKRI,
SEQ ID NO: 24
SVYAWNRKRISNCVADYSVLYNSAS,
SEQ ID NO: 25
DYSVLYNSASFSTFKCYGVSPTKLN,
SEQ ID NO: 26
CYGVSPTKLNDLCFTNVYADSFVIR,
SEQ ID NO: 27
NVYADSFVIRGDEVRQIAPGQTGKI,
SEQ ID NO: 28
RQIAPGQTGKIADYNYKLPDDFTGC,
SEQ ID NO: 29
YKLPDDFTGCVIAWNSNNLDSKVGG,
SEQ ID NO: 30
SNNLDSKVGGNYNYLYRLFRKSNLK,
SEQ ID NO: 31
YRLFRKSNLKPFERDISTEIYQAGS,
SEQ ID NO: 32
ISTEIYQAGSTPCNGVEGFNCYFPL,
SEQ ID NO: 33
VEGFNCYFPLQSYGFQPTNGVGYQP,
SEQ ID NO: 34
FQPTNGVGYQPYRVVVLSFELLHAP,
SEQ ID NO: 35
VLSFELLHAPATVCGPKKSTNLVKN,
and
SEQ ID NO: 36
PKKSTNLVKNKCVNF,
or one or more variants thereof having at least 80% amino acid sequence identity to the amino acid sequences set forth in SEQ ID NOS:22-36.

3. The composition of claim 1, comprising a first set of 21 isolated oligopeptides that comprise the amino acid sequences set forth in SEQ ID NOS: 1-21 or that comprise the amino acid sequences set forth in SEQ ID NOS: 1-20 and 39, or one or more variants thereof having at least 80% amino acid sequence identity to the amino acid sequences set forth in one or more of SEQ ID NOS:1-21 or in SEQ ID NOS: 1-20 and 39.

4. The composition of claim 3 which further comprises a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

5. A composition for diagnosis or prognosis of coronavirus disease 2019 (Covid-19), or for detecting an antigen-specific T cell-mediated immune response to severe acute respiratory syndrome coronavirus 2 (SARS-CoV-2), comprising:
(a) 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36, or one or more variants thereof having at least 80% amino acid sequence identity to the amino acid sequences set forth in SEQ ID NOS:22-36; or
(b) a set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS:22-36, and that each comprise a SARS-CoV-2 Spike protein receptor binding domain (RBD) CD4+ T-cell epitope.

6. A method for detecting SARS-CoV-2 spike protein antigen-specific cell-mediated immune response activity in a biological sample from a subject, comprising
(a) incubating in vitro an incubation test mixture that comprises (i) a biological sample comprising T-cells and antigen-presenting cells from the subject admixed and (ii) a first peptide composition comprising a first set of 21 isolated oligopeptides that comprise the amino acid sequences set forth in SEQ ID NOS: 1-21 or that comprise the amino acid sequences set forth in SEQ ID NOS: 1-20 and 39, or one or more variants thereof having at least 80% amino acid sequence identity to the amino acid sequences set forth in one or more of SEQ ID NOS:1-21 or in one or more of SEQ ID NOS: 1-20 and 39, under conditions and for a time sufficient for specific recognition by said T-cells of a SARS-CoV-2 spike protein T-cell epitope that is present in said first composition to stimulate generation of a T-cell immune response indicator; and
(b) detecting a first level of the T-cell immune response indicator in the incubation test mixture, wherein presence of SARS-CoV-2 spike protein antigen-specific cell-mediated immune response activity in the biological sample is indicated by detection in (b) of said first level of the T-cell immune response indicator that is increased relative to a control level of the T-cell immune response indicator obtained by incubating the biological sample in a control incubation without the peptide composition,
and thereby detecting SARS-CoV-2 spike protein antigen-specific cell-mediated immune response activity.

7. The method of claim 6 wherein the incubation test mixture further comprises (iii) a second peptide composition comprising a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

8. The method of claim 6 wherein the biological sample is obtained from the subject before, after, or before and after a SARS-CoV-2 vaccine has been administered to the subject.

9. The method of claim 6 wherein the biological sample comprises at least one of whole blood, sputum, pulmonary lavage fluid, or lymph.

10. The method of claim 6 wherein the biological sample comprises at least one of (a) whole blood, (b) a cellular fraction of whole blood, (c) isolated peripheral blood white cells, or (d) isolated peripheral blood mononuclear cells.

11. The method of claim 6 wherein the T-cell immune response indicator is interferon-gamma (IFN-γ).

12. The method of claim 11 wherein the IFN-γ is soluble IFN-γ released by the T-cells.

13. The method of claim 6 wherein the T-cell immune response indicator comprises at least one of T-cell proliferation and expression of a T-cell cytokine.

14. The method of claim 13 wherein the T-cell cytokine is selected from IL-1α, IL-1β, IL-2, IL-10, IL-12, IL-17, TNF-α, TNF-β, and IFN-γ.

15. The method of claim 13 wherein expression of the T-cell cytokine is detected as soluble T-cell cytokine released by the T-cells.

16. The method of claim 15 wherein the T-cell cytokine is selected from IL-1α, IL-1β, IL-2, IL-10, IL-12, IL-17, TNF-α, TNF-β, and IFN-γ.

17. The method of claim 16 wherein the T-cell cytokine is detected by determining detectable specific binding of a binding agent to the T-cell cytokine.

18. The method of claim 17 wherein the binding agent comprises at least one antibody that binds specifically to the T-cell cytokine.

19. The method of claim 18 wherein the at least one antibody is selected from a monoclonal antibody and a polyclonal antibody.

20. The method of claim 18 wherein the at least one antibody is immobilized on a solid phase.

21. A composition that is selected from a first nucleic acid composition and a second nucleic acid composition:
(I) the first nucleic acid composition comprising one or a plurality of isolated nucleic acid molecules that encode 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 or in one of SEQ ID NOS: 1-20 and 39, or one or more variants thereof having at least 80% amino acid sequence identity to the amino acid sequences set forth in SEQ ID NOS:1-21 or in SEQ ID NOS: 1-20 and 39, wherein the isolated oligopeptides, after being contacted with a whole blood sample obtained from a subject who has previously been infected with SARS-CoV-2, are capable of eliciting a secondary in vitro immune response by T-cells in the whole blood sample; and
(II) the second nucleic acid composition comprising one or a plurality of isolated nucleic acid molecules that encode 21 isolated oligopeptides that comprise the amino acid sequences set forth in SEQ ID NOS: 1-21 or that comprise the amino acid sequences set forth in SEQ ID NOS: 1-20 and 39, or one or more variants thereof having at least 80% amino acid sequence identity to the amino acid sequences set forth in one or more of SEQ ID NOS:1-21 or in one or more of SEQ ID NOS: 1-20 and 39, wherein the isolated oligopeptides, after being contacted with a whole blood sample obtained from a subject who has previously been infected with SARS-CoV-2, are capable of eliciting a secondary in vitro immune response by T-cells in the whole blood sample.

22. A composition that is selected from a first nucleic acid composition and a second nucleic acid composition:
(I) the first nucleic acid composition comprising one or a plurality of isolated nucleic acid molecules that encode 1, 2, 3, 4, 5, 6, 7, 8, 9, 10, 11, 12, 13, 14, or 15 isolated oligopeptides that each comprise a SARS-CoV-2 Spike protein receptor binding domain (RBD) CD4+ T-cell epitope comprising the amino acid sequence set forth in one of SEQ ID NOS: 22-36 or one or more variants thereof having at least 80% amino acid sequence identity to the amino acid sequences set forth in SEQ ID NOS:22-36, wherein the isolated oligopeptides, after being contacted with a whole blood sample obtained from a subject who has previously been infected with SARS-CoV-2, are capable of eliciting a secondary in vitro immune response by T-cells in the whole blood sample; and
(II) the second nucleic acid composition comprising one or a plurality of isolated nucleic acid molecules that encode 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS:22-36, wherein the isolated oligopeptides, after being contacted with a whole blood sample obtained from a subject who has previously been infected with SARS-CoV-2, are capable of eliciting a secondary in vitro immune response by T-cells in the whole blood sample.

23. A vector composition comprising one or more nucleic acid vectors that comprise the composition of claim 21.

24. A host cell comprising the vector composition of claim 23.

25. The composition of claim 1 wherein the 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprise at least:
(a) the amino acid sequences set forth in SEQ ID NOS: 1, 3, and 5, or one or more variants having at least 80% amino acid sequence identity thereto;
(b) the amino acid sequences set forth in SEQ ID NOS: 2, 10, and 12, or one or more variants having at least 80% amino acid sequence identity thereto;
(c) the amino acid sequences set forth in SEQ ID NOS: 3, 6, and 11, or one or more variants having at least 80% amino acid sequence identity thereto; or
(d) the amino acid sequences set forth in SEQ ID NOS: 4, 19, and 20, or one or more variants having at least 80% amino acid sequence identity thereto.

26. The composition of claim 1 wherein the 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprise at least:
(a) the amino acid sequences set forth in SEQ ID NOS: 1, 3, and 5, or one or more variants having at least 80% amino acid sequence identity thereto;
(b) the amino acid sequences set forth in SEQ ID NOS: 2, 4, and 7, or one or more variants having at least 80% amino acid sequence identity thereto;
(c) the amino acid sequences set forth in SEQ ID NOS: 8, 10, and 12, or one or more variants having at least 80% amino acid sequence identity thereto;
(d) the amino acid sequences set forth in SEQ ID NOS: 9, 14, and 15, or one or more variants having at least 80% amino acid sequence identity thereto;
(e) the amino acid sequences set forth in SEQ ID NOS: 6, 11, and 18, or one or more variants having at least 80% amino acid sequence identity thereto;
(f) the amino acid sequences set forth in SEQ ID NOS: 13, 16, and 39, or one or more variants having at least 80% amino acid sequence identity thereto; or
(g) the amino acid sequences set forth in SEQ ID NOS: 17, 19, and 20, or one or more variants having at least 80% amino acid sequence identity thereto.

27. A method for detecting SARS-CoV-2 spike protein antigen-specific cell-mediated immune response activity in a biological sample from a subject, comprising
(a) incubating in vitro an incubation test mixture that comprises (i) a biological sample comprising T-cells and antigen-presenting cells from the subject admixed and (ii) a first peptide composition comprising 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprising the amino acid sequence set forth in one of SEQ ID NOS: 1-21 or in one of SEQ ID NOS: 1-20 and 39, or one or more variants thereof having at least 80% amino acid sequence identity to the amino acid sequences set forth in SEQ ID NOS:1-21 or in SEQ ID NOS: 1-20 and 39, under conditions and for a time sufficient for specific recognition by said T-cells of a SARS-CoV-2 spike protein T-cell epitope that is present in said first composition to stimulate generation of a T-cell immune response indicator; and
(b) detecting a first level of the T-cell immune response indicator in the incubation test mixture, wherein presence of SARS-CoV-2 spike protein antigen-specific cell-mediated immune response activity in the biological sample is indicated by detection in (b) of said first level of the T-cell immune response indicator that is increased relative to a control level of the T-cell immune response indicator obtained by incubating the biological sample in a control incubation without the peptide composition,
and thereby detecting SARS-CoV-2 spike protein antigen-specific cell-mediated immune response activity.

28. The method of claim 27 wherein the incubation test mixture further comprises (iii) a second peptide composition comprising a second set of 15 isolated oligopeptides that comprise the amino acid sequences set forth in SEQ ID NOS: 22-36, or one or more variants thereof having at least 80% amino acid sequence identity to the amino acid sequences set forth in one or more of SEQ ID NOS: 22-36.

29. The method of claim 27 wherein the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprises at least:
(a) the amino acid sequences set forth in SEQ ID NOS: 1, 3, and 5 or one or more variants having at least 80% amino acid sequence identity thereto;
(b) the amino acid sequences set forth in SEQ ID NOS: 2, 10, and 12 or one or more variants having at least 80% amino acid sequence identity thereto;
(c) the amino acid sequences set forth in SEQ ID NOS: 3, 6, and 11 or one or more variants having at least 80% amino acid sequence identity thereto; or
(d) the amino acid sequences set forth in SEQ ID NOS: 4, 19, and 20 or one or more variants having at least 80% amino acid sequence identity thereto.

30. The method of claim 27 wherein the first peptide composition of 1, 2, 3, 4, 5, 6, 7, 8, 9, 10, 11, 12, 13, 14, 15, 16, 17, 18, 19, 20, or 21 isolated oligopeptides that each comprise a SARS-CoV-2 Spike protein S1 or S2 region CD8+ T-cell epitope comprises at least:
(a) the amino acid sequences set forth in SEQ ID NOS: 1, 3, and 5 or one or more variants having at least 80% amino acid sequence identity thereto;
(b) the amino acid sequences set forth in SEQ ID NOS: 2, 4, and 7 or one or more variants having at least 80% amino acid sequence identity thereto;
(c) the amino acid sequences set forth in SEQ ID NOS: 8, 10, and 12 or one or more variants having at least 80% amino acid sequence identity thereto;
(d) the amino acid sequences set forth in SEQ ID NOS: 9, 14, and 15 or one or more variants having at least 80% amino acid sequence identity thereto;
(e) the amino acid sequences set forth in SEQ ID NOS: 6, 11, and 18 or one or more variants having at least 80% amino acid sequence identity thereto;
(f) the amino acid sequences set forth in SEQ ID NOS: 13, 16, and 39 or one or more variants having at least 80% amino acid sequence identity thereto; or
(g) the amino acid sequences set forth in SEQ ID NOS: 17, 19, and 20 or one or more variants having at least 80% amino acid sequence identity thereto.

31. The method of claim 27 wherein the biological sample is obtained from the subject before, after, or before and after a SARS-CoV-2 vaccine has been administered to the subject.

32. The method of claim 27 wherein the biological sample comprises at least one of whole blood, sputum, pulmonary lavage fluid, or lymph.

33. The method of claim 27 wherein the biological sample comprises at least one of (a) whole blood, (b) a cellular fraction of whole blood, (c) isolated peripheral blood white cells, or (d) isolated peripheral blood mononuclear cells.

34. The method of claim 27 wherein the T-cell immune response indicator is interferon-gamma (IFN-γ).

35. The method of claim 34 wherein the IFN-γ is soluble IFN-γ released by the T-cells.

36. The method of claim 27 wherein the T-cell immune response indicator comprises at least one of T-cell proliferation and expression of a T-cell cytokine.

37. The method of claim 36 wherein the T-cell cytokine is selected from IL-1α, IL-1β, IL-2, IL-10, IL-12, IL-17, TNF-α, TNF-β, and IFN-γ.

38. The method of claim 36 wherein expression of the T-cell cytokine is detected as soluble T-cell cytokine released by the T-cells.

39. The method of claim 38 wherein the T-cell cytokine is selected from IL-1α, IL-1β, IL-2, IL-10, IL-12, IL-17, TNF-α, TNF-β, and IFN-γ.

40. The method of claim 36 wherein the T-cell cytokine is detected by determining detectable specific binding of a binding agent to the T-cell cytokine.

41. The method of claim 40 wherein the binding agent comprises at least one antibody that binds specifically to the T-cell cytokine.

42. The method of claim 41 wherein the at least one antibody is selected from a monoclonal antibody and a polyclonal antibody.

43. The method of claim 41 wherein the at least one antibody is immobilized on a solid phase.


SEQUENCE LISTING (39 sequences)

SEQ ID NO: 1 - protein, 14 aa, Artificial Sequence
  SARS-CoV-2 Spike protein S1 or S2 region CD8+ T-cell epitope
YPDKVFRSSVLHST

SEQ ID NO: 2 - protein, 13 aa, Artificial Sequence
  SARS-CoV-2 Spike protein S1 or S2 region CD8+ T-cell epitope
VLHSTQDLFLPFF

SEQ ID NO: 3 - protein, 11 aa, Artificial Sequence
  SARS-CoV-2 Spike protein S1 or S2 region CD8+ T-cell epitope
KSWMESEFRVY

SEQ ID NO: 4 - protein, 13 aa, Artificial Sequence
  SARS-CoV-2 Spike protein S1 or S2 region CD8+ T-cell epitope
RVYSSANNCTFEY

SEQ ID NO: 5 - protein, 12 aa, Artificial Sequence
  SARS-CoV-2 Spike protein S1 or S2 region CD8+ T-cell epitope
EFVFKNIDGYFK

SEQ ID NO: 6 - protein, 15 aa, Artificial Sequence
  SARS-CoV-2 Spike protein S1 or S2 region CD8+ T-cell epitope
YYVGYLQPRTFLLKY

SEQ ID NO: 7 - protein, 14 aa, Artificial Sequence
  SARS-CoV-2 Spike protein S1 or S2 region CD8+ T-cell epitope
EVFNATRFASVYAW

SEQ ID NO: 8 - protein, 14 aa, Artificial Sequence
  SARS-CoV-2 Spike protein S1 or S2 region CD8+ T-cell epitope
RISNCVADYSVLYN

SEQ ID NO: 9 - protein, 15 aa, Artificial Sequence
  SARS-CoV-2 Spike protein S1 or S2 region CD8+ T-cell epitope
YSVLYNSASFTFKCY

SEQ ID NO: 10 - protein, 11 aa, Artificial Sequence
  SARS-CoV-2 Spike protein S1 or S2 region CD8+ T-cell epitope
CFTNVYADSFV

SEQ ID NO: 11 - protein, 13 aa, Artificial Sequence
  SARS-CoV-2 Spike protein S1 or S2 region CD8+ T-cell epitope
LYRLFRKSNLKPF

SEQ ID NO: 12 - protein, 13 aa, Artificial Sequence
  SARS-CoV-2 Spike protein S1 or S2 region CD8+ T-cell epitope
YQPYRVVVLSFEL

SEQ ID NO: 13 - protein, 12 aa, Artificial Sequence
  SARS-CoV-2 Spike protein S1 or S2 region CD8+ T-cell epitope
WRVYSTGSNVFQ

SEQ ID NO: 14 - protein, 15 aa, Artificial Sequence
  SARS-CoV-2 Spike protein S1 or S2 region CD8+ T-cell epitope
TNSPRRARSVASQSI

SEQ ID NO: 15 - protein, 15 aa, Artificial Sequence
  SARS-CoV-2 Spike protein S1 or S2 region CD8+ T-cell epitope
RSVASQSIIAYTMSL

SEQ ID NO: 16 - protein, 11 aa, Artificial Sequence
  SARS-CoV-2 Spike protein S1 or S2 region CD8+ T-cell epitope
MTKTSVDCTMY

SEQ ID NO: 17 - protein, 16 aa, Artificial Sequence
  SARS-CoV-2 Spike protein S1 or S2 region CD8+ T-cell epitope
PLLTDEMIAQYTSALL

SEQ ID NO: 18 - protein, 15 aa, Artificial Sequence
  SARS-CoV-2 Spike protein S1 or S2 region CD8+ T-cell epitope
AALQIPFAMQMAYRF

SEQ ID NO: 19 - protein, 16 aa, Artificial Sequence
  SARS-CoV-2 Spike protein S1 or S2 region CD8+ T-cell epitope
RAAEIRASANLAATKM

SEQ ID NO: 20 - protein, 17 aa, Artificial Sequence
  SARS-CoV-2 Spike protein S1 or S2 region CD8+ T-cell epitope
KYEQYIKWPWYIWLGFI

SEQ ID NO: 21 - protein, 15 aa, Artificial Sequence
  SARS-CoV-2 Spike protein S1 or S2 region CD8+ T-cell epitope
YIWLGFIAGLIAIVM

SEQ ID NO: 22 - protein, 25 aa, Artificial Sequence
  SARS-CoV-2 Spike protein receptor binding domain (RBD) CD4+ T-cell epitope
RVQPTESIVRFPNITNLCPFGEVFN

SEQ ID NO: 23 - protein, 25 aa, Artificial Sequence
  SARS-CoV-2 Spike protein receptor binding domain (RBD) CD4+ T-cell epitope
NLCPFGEVFNATRFASVYAWNRKRI

SEQ ID NO: 24 - protein, 25 aa, Artificial Sequence
  SARS-CoV-2 Spike protein receptor binding domain (RBD) CD4+ T-cell epitope
SVYAWNRKRISNCVADYSVLYNSAS

SEQ ID NO: 25 - protein, 25 aa, Artificial Sequence
  SARS-CoV-2 Spike protein receptor binding domain (RBD) CD4+ T-cell epitope
DYSVLYNSASFSTFKCYGVSPTKLN

SEQ ID NO: 26 - protein, 25 aa, Artificial Sequence
  SARS-CoV-2 Spike protein receptor binding domain (RBD) CD4+ T-cell epitope
CYGVSPTKLNDLCFTNVYADSFVIR

SEQ ID NO: 27 - protein, 25 aa, Artificial Sequence
  SARS-CoV-2 Spike protein receptor binding domain (RBD) CD4+ T-cell epitope
NVYADSFVIRGDEVRQIAPGQTGKI

SEQ ID NO: 28 - protein, 25 aa, Artificial Sequence
  SARS-CoV-2 Spike protein receptor binding domain (RBD) CD4+ T-cell epitope
RQIAPGQTGKIADYNYKLPDDFTGC

SEQ ID NO: 29 - protein, 25 aa, Artificial Sequence
  SARS-CoV-2 Spike protein receptor binding domain (RBD) CD4+ T-cell epitope
YKLPDDFTGCVIAWNSNNLDSKVGG

SEQ ID NO: 30 - protein, 25 aa, Artificial Sequence
  SARS-CoV-2 Spike protein receptor binding domain (RBD) CD4+ T-cell epitope
SNNLDSKVGGNYNYLYRLFRKSNLK

SEQ ID NO: 31 - protein, 25 aa, Artificial Sequence
  SARS-CoV-2 Spike protein receptor binding domain (RBD) CD4+ T-cell epitope
YRLFRKSNLKPFERDISTEIYQAGS

SEQ ID NO: 32 - protein, 25 aa, Artificial Sequence
  SARS-CoV-2 Spike protein receptor binding domain (RBD) CD4+ T-cell epitope
ISTEIYQAGSTPCNGVEGFNCYFPL

SEQ ID NO: 33 - protein, 25 aa, Artificial Sequence
  SARS-CoV-2 Spike protein receptor binding domain (RBD) CD4+ T-cell epitope
VEGFNCYFPLQSYGFQPTNGVGYQP

SEQ ID NO: 34 - protein, 25 aa, Artificial Sequence
  SARS-CoV-2 Spike protein receptor binding domain (RBD) CD4+ T-cell epitope
FQPTNGVGYQPYRVVVLSFELLHAP

SEQ ID NO: 35 - protein, 25 aa, Artificial Sequence
  SARS-CoV-2 Spike protein receptor binding domain (RBD) CD4+ T-cell epitope
VLSFELLHAPATVCGPKKSTNLVKN

SEQ ID NO: 36 - protein, 15 aa, Artificial Sequence
  SARS-CoV-2 Spike protein receptor binding domain (RBD) CD4+ T-cell epitope
PKKSTNLVKNKCVNF

SEQ ID NO: 37 - protein, 1273 aa, Artificial Sequence
  SARS-CoV-2 Spike glycoprotein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - protein, 223 aa, Artificial Sequence
  Receptor Binding Domain (RBD) of SARS-CoV-2 Spike glycoprotein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 39 - protein, 12 aa, Artificial Sequence
  SARS-CoV-2 Spike protein S1 or S2 region CD8+ T-cell epitope
YHLMSFPQSAPH